(19) United States
(12) Patent Application Publication
(10) Pub. No.: US 2024/0000344 A1
(43) Pub. Date: Jan. 4, 2024

(54) SYSTEMS AND METHODS FOR IDENTIFYING USER BODY POSITION DURING RESPIRATORY THERAPY

(71) Applicants: ResMed Sensor Technologies Limited, Dublin (IE); ResMed Pty Ltd., Bella Vista, NSW (AU)

(72) Inventors: LUCA CERINA, DUBLIN (IE); WADUGE VARUNI LAKSHANA FERNANDO, DUBLIN (IE); Liam Holley, Sydney (AU)

(21) Appl. No.: 18/039,093

(22) PCT Filed: Nov. 26, 2021
(86) PCT No.: PCT/IB2021/061026

§ 371 Date: May 26, 2023

Related U.S. Application Data
(60) Provisional application No. 63/118,848, filed on Nov. 27, 2020.

(51) Int. Cl. A61B 5/11; A61B 5/087; A61B 5/00

(52) U.S. Cl. CPC A61B 5/1126; A61B 5/087; A61B 5/4818

(57) ABSTRACT

A system for identifying a body position of a user of a respiratory therapy system includes a sensor, a memory, and a control system. The sensor is configured to generate airflow data associated with the user. The memory stores machine-readable instructions. The control system includes one or more processors configured to execute the machine-readable instructions to receive the airflow data associated with the user during a sleep session. The control system is further configured to determine one or more features associated with the airflow data, and identify the body position of the user during a first portion of the sleep session based at least in part on the determined one or more features. The control system is further configured to cause an action to be performed based at least in part on the identified body position of the user.

### CROSS-REFERENCE TO RELATED APPLICATIONS

This application claims the benefit of, and priority to, U.S. Provisional Patent Application No. 63/118,848 filed on Nov. 27, 2020, which is hereby incorporated by reference herein in its entirety.

### TECHNICAL FIELD

The present disclosure relates generally to improving user experience in respiratory therapy, and more particularly, to systems and methods for identifying user body position during respiratory therapy.

### BACKGROUND

Various systems exist for aiding users experiencing sleep apnea and related respiratory disorders. A range of respiratory disorders exist that can impact users. Certain disorders are characterized by particular events (e.g., apneas, hypopneas, hyperpneas, or any combination thereof). Examples of respiratory disorders include periodic limb movement disorder (PLMD), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and Chest wall disorders.

Studies on large cohorts of users show a high prevalence of positional Obstructive Sleep Apnea (POSA) and exclusive POSA (ePOSA, sleep apneas only in supine position) of 75% and 36% of OSA users. Positional therapy not only can provide treatment for users with mild OSA, but also for users undergoing respiratory therapy who could have a more comfortable option and/or could improve their respiratory therapy. User body position can be used for adjusting respiratory therapy. Thus, a need exists for systems and methods for identifying body position of users undergoing respiratory therapy. The present disclosure is directed to solving these and other problems.

### SUMMARY

According to some implementations of the present disclosure, a system for identifying a body position of a user of a respiratory therapy system is disclosed as follows. The system includes a sensor, a memory, and a control system. The sensor is configured to generate airflow data associated with the user. The memory stores machine-readable instructions. The control system includes one or more processors configured to execute the machine-readable instructions to receive the airflow data associated with the user during a sleep session. The control system is further configured to determine one or more features associated with the airflow data. The control system is further configured to identify the body position of the user during a first portion of the sleep session based at least in part on the determined one or more features. The control system is further configured to cause an action to be performed based at least in part on the identified body position of the user.

According to some implementations of the present disclosure, a system for identifying a movement event of a user of a respiratory therapy system is disclosed as follows. The system includes a sensor, a memory, and a control system. The sensor is configured to generate airflow data associated with the user. The memory stores machine-readable instructions. The control system includes one or more processors configured to execute the machine-readable instructions to receive the airflow data associated with the user during a sleep session. The control system is further configured to determine one or more features associated with the airflow data. The control system is further configured to identify the movement event of the user during a first portion of the sleep session based at least in part on the determined one or more features. The control system is further configured to cause an action to be performed based at least in part on the identified movement event of the user.

According to some implementations of the present disclosure, a method for identifying a body position of a user of a respiratory therapy system during a sleep session is disclosed as follows. Airflow data associated with the user of the respiratory therapy system during the sleep session is received. One or more features associated with the airflow data are determined. Based at least in part on the determined one or more features, the body position of the user during a first portion of the sleep session is identified. Based at least in part on the identified body position of the user, an action is caused to be performed.

According to some implementations of the present disclosure, a system includes a control system and a memory. The control system includes one or more processors. The memory has stored thereon machine readable instructions. The control system is coupled to the memory. Any one of the methods disclosed herein is implemented when the machine executable instructions in the memory are executed by at least one of the one or more processors of the control system.

According to some implementations of the present disclosure, a system for identifying a body position of a user of a respiratory therapy system during a sleep session is disclosed as follows. The system includes a control system configured to implement any one of the methods disclosed herein.

According to some implementations of the present disclosure, a computer program product comprising instructions which, when executed by a computer, cause the computer to carry out any one of the methods disclosed herein. In some implementations, the computer program product is a non-transitory computer readable medium.

The foregoing and additional aspects and implementations of the present disclosure will be apparent to those of ordinary skill in the art in view of the detailed description of various embodiments and/or implementations, which is made with reference to the drawings, a brief description of which is provided next.

### BRIEF DESCRIPTION OF THE DRAWINGS

The foregoing and other advantages of the present disclosure will become apparent upon reading the following detailed description and upon reference to the drawings.

FIG. 1 is a functional block diagram of a system for identifying a body position of a user of a respiratory therapy system, according to some implementations of the present disclosure.

FIG. 2 is a perspective view of at least a portion of the system of FIG. 1, a user wearing a full face mask, and a bed partner, according to some implementations of the present disclosure.

FIG. 3A illustrates a breath waveform of an individual while sleeping, according to some implementations of the present disclosure.

FIG. 3B illustrates selected polysomnography channels (pulse oximetry, flow rate, thoracic movement, and abdominal movement) of an individual during non-REM sleep breathing normally over a period of about ninety seconds, according to some implementations of the present disclosure.

FIG. 3C illustrates polysomnography of an individual before respiratory treatment, according to some implementations of the present disclosure.

FIG. 3D illustrates flow rate data where an individual is experiencing a series of total obstructive apneas, according to some implementations of the present disclosure.

FIG. 4A illustrates flow rate data associated with a user of a respiratory therapy system, according to some implementations of the present disclosure.

FIG. 4B illustrates pressure data associated with a user of a respiratory therapy system, according to some implementations of the present disclosure.

FIG. 4C illustrates pressure data associated with a user of a respiratory therapy system with an expiratory pressure relief module, according to some implementations of the present disclosure.

FIG. 5 illustrates positional data and flow rate data associated with a user of a respiratory therapy system, according to some implementations of the present disclosure.

FIG. 6 illustrates analyzed positional data and features derived from flow rate data associated with a user of a respiratory therapy system, according to some implementations of the present disclosure.

FIG. 7 illustrates analyzed positional data, features derived from flow rate data, and apnea/hypopnea data associated with a user of a respiratory therapy system, according to some implementations of the present disclosure.

FIG. 8 illustrates positional data and flow rate data associated with a user of a respiratory therapy system during a first portion of a sleep session, according to some implementations of the present disclosure.

FIG. 9 illustrates positional data and flow rate data associated with a user of a respiratory therapy system, according to some implementations of the present disclosure.

FIG. 10 illustrates positional data and flow rate data associated with a user of a respiratory therapy system during a first portion of a sleep session, according to some implementations of the present disclosure.

FIG. 11 illustrates analyzed positional data, features derived from flow rate data, and apnea/hypopnea data associated with a user of a respiratory therapy system, according to some implementations of the present disclosure.

FIG. 12 illustrates analyzed positional data, features derived from flow rate data, and apnea/hypopnea data associated with the user of FIG. 11, according to some implementations of the present disclosure.

FIG. 13 illustrates analyzed positional data, features derived from flow rate data, and apnea/hypopnea data associated with a user of a respiratory therapy system, according to some implementations of the present disclosure.

FIG. 14 illustrates analyzed positional data, features derived from flow rate data, and apnea/hypopnea data associated with the user of FIG. 13, according to some implementations of the present disclosure.

FIG. 15 illustrates analyzed positional data, features derived from flow rate data, and apnea/hypopnea data associated with a user of a respiratory therapy system, according to some implementations of the present disclosure.

FIG. 16 illustrates analyzed positional data, features derived from flow rate data, and apnea/hypopnea data associated with the user of FIG. 15, according to some implementations of the present disclosure.

FIG. 17 illustrates analyzed positional data, features derived from flow rate data, and apnea/hypopnea data associated with a user of a respiratory therapy system, according to some implementations of the present disclosure.

FIG. 18 illustrates analyzed positional data, features derived from flow rate data, and apnea/hypopnea data associated with the user of FIG. 18, according to some implementations of the present disclosure.

FIG. 19 illustrates a flow diagram for a method identifying a body position of a user of a respiratory therapy system during a sleep session, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hyper tension, diabetes, stroke, insomnia, and chest wall disorders.

Obstructive Sleep Apnea (OSA) is a form of Sleep Disordered Breathing (SDB), and is characterized by events including occlusion or obstruction of the upper air passage during sleep, often resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Typically, the individual will stop breathing for between about 15 seconds and about 30 seconds during an obstructive sleep apnea event.

Other types of events include hypopnea, hyperpnea, and hypercapnia. Hypopnea is generally characterized by slow or shallow breathing caused by a narrowed airway, as opposed to a blocked airway. Both apnea and hypopnea are characterized by an accompanying reduction in oxygen saturation in the bloodstream. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in its entirety.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the total number of apnea and hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea. Particularly in the case of lower AHI levels that might be indicative of mild sleep apnea, it is also desirable to assess the severity of daytime symptoms, such as excessive daytime sleepiness, in order to gain a more complete picture of the severity of the disease.

Everyone has their own preferences for sleeping. Whether it's sleeping completely flat (e.g., in a horizontal position), reclined, or sitting upright; or whether it's lying on their stomach (e.g., in a prone position), on their back (in a supine position), or on your left or right side. For most normal people, their body position will change many times during a sleep session. For example, an individual will normally arouse briefly from sleep and change positions before entering sleep again in the new position. For young adults, this shifting of position during sleep may happen in the order of four to five shifts per hour. As humans age, the number of shifts in sleep position per sleep session tends to reduce, such that elderly people more commonly might only shift sleeping positions twice per hour on average. In addition, younger adults and children are expected to experience similar durations in the various trunk positions, whereas more elderly people tend to spend less time in the prone position and more time supine.

Breathing conditions for an individual's body are different when the individual is lying down as compared to when the individual is standing up. When the individual is sitting or is on the feet, the individual's airway is pointing downward, leaving breathing and airflow relatively unrestricted. However, when the individual settles down to sleep, the individual's body is imposed to breathing in a substantially horizontal position, meaning that gravity is now working against the airway. Sleep apnea and snoring can occur when the muscular tissues in the top airway relax and the individual's lungs get limited air to breathe via the nose or throat. While the process of breathing is the same at night, the individual's surrounding tissues can vibrate, causing the individual to snore. Even relaxed muscles can cause sleep apnea because the total blockage of the airway hampers breathing fully, forcing the individual to wake up in the middle of sleep. As a result, there are advantages afforded to the individual for sleeping in positions that best support the individual's breathing patterns. For example, some individual may benefit from sleeping in a reclined position rather than completely horizontal relative to ground.

Sleeping in the supine position can often be problematic for those who have snoring problems, breathing problems, or sleep apnea. This happens because the gravitational force enhances the capacity of the jaw, the tongue, and soft palate to drop back toward the throat. It narrows the airways and can cause increased resistance to airflow while breathing.

Sleeping in the prone position may seem like an alternative to the gravity issue as the downward force pulls the tongue and palate forward. While this is true to an extent, when sleeping in this position, the individual's nose and mouth can become blocked by the pillow. It may affect the individual's breathing. Apart from this, it may also cause neck pain, cervical problems, or digestion problems, which in turn affect the individual's sleep quality.

Some studies suggest that sleeping on the side may be the most ideal position for snoring and sleep apnea sufferers. Because when the individual's body is positioned on its side during rest, the airways are more stable and less likely to collapse or restrict air. In this position, the individual's body, head and torso are positioned on one side (left or right), arms are under the body or a bit forward or extended, and legs are packed with one under the other or slightly staggered. While both lateral (left and right) sides are considered as good sleeping positions, for some the left lateral position may not be an ideal one. That's because while sleeping on the left side, the internal organs of the body in the thorax can face some movement. And the lungs may add more weight or pressure on the heart. This can affect the heart's function, and it can retaliate by activating the kidneys, causing an increased need for urination at night. The right side, however, puts less pressure on the vital organs, such as lungs and heart. Sleeping on a particular side can also be ideal if a joint (often shoulder or hip) on the individual's other side is causing pain.

When an individual has sleep apnea or other breathing disorders, getting a good and peaceful sleep becomes difficult. However, choosing the right sleeping position can help the user get comfortable and at the same time help overcome the breathing problems that the individual usually face while sleeping. Thus, according to some implementations of the present disclosure, systems and methods are provided to create a personalized model for identifying a user's body position during respiratory therapy. Positional therapy not only can provide treatment for users with mild OSA, but also for users already undergoing another therapy who could have a more comfortable option (e.g., lower pressure in CPAP, smaller displacement in mandibular repositioning devices, etc.).

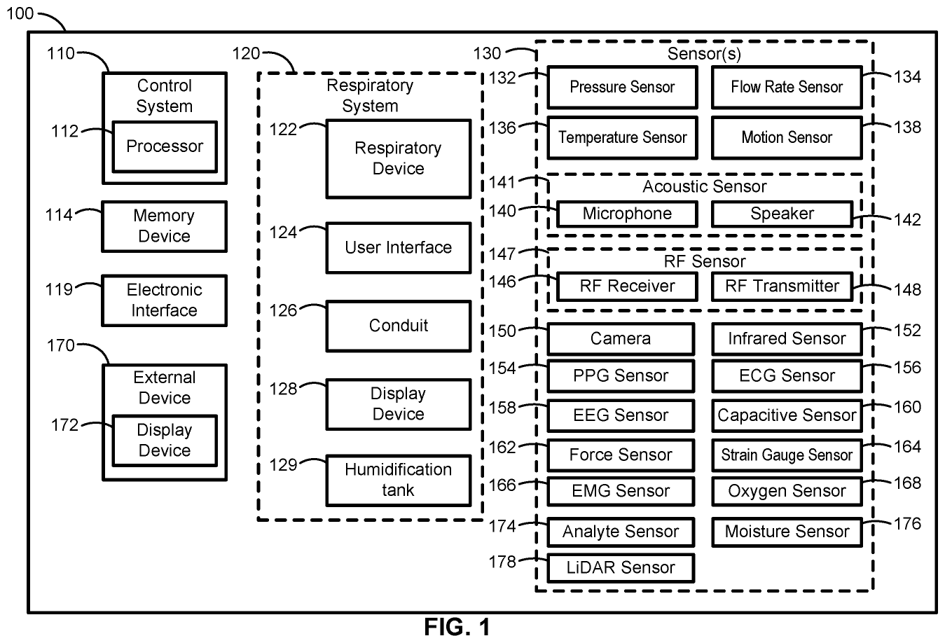

Referring to FIG. 1, a system 100, according to some implementations of the present disclosure, is illustrated. The system 100 may be for providing a variety of different sensors related to a user's use of a respiratory therapy system, among other uses. The system 100 includes a control system 110, a memory device 114, an electronic interface 119, one or more sensors 130, and one or more user devices 170. In some implementations, the system 100 further includes a respiratory therapy system 120 that includes a respiratory therapy device 122. The system 100 can be used to identify a body position of a user while using the respiratory therapy system 120, as disclosed in further detail herein.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, a portion (e.g., a housing) of the respiratory therapy system 120, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) test result or score and/or a Pittsburgh Sleep Quality Index (PSQI) score or value. The medical information data can include results from one or more of a polysomnography (PSG) test, a CPAP titration, or a home sleep test (HST), respiratory therapy system settings from one or more sleep sessions, sleep related respiratory events from one or more sleep sessions, or any combination thereof. The self-reported user feedback can include information indicative of a self-reported subjective therapy score (e.g., poor, average, excellent), a self-reported subjective stress level of the user, a self-reported subjective fatigue level of the user, a self-reported subjective health status of the user, a recent life event experienced by the user, or any combination thereof. In some implementations, the memory device 114 stores media content that can be displayed on the display device 128 and/or the display device 172.

The electronic interface 119 is configured to receive data (e.g., physiological data and/or flow rate data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a Wi-Fi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

The respiratory therapy system 120 can include a respiratory pressure therapy (RPT) device 122 (referred to herein as respiratory therapy device 122), a user interface 124, a conduit 126 (also referred to as a tube or an air circuit), a display device 128, a humidification tank 129 or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 is generally used to generate pressurized air that is delivered to a user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory therapy device 122, the user interface 124, and the conduit 126 form an air pathway fluidly coupled with an airway of the user. The pressurized air also increases the user's oxygen intake during sleep.

Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cmH₂O relative to ambient pressure. For other forms of therapy, such as the delivery of oxygen, the user interface may not include a seal sufficient to facilitate delivery to the airways of a supply of gas at a positive pressure of about 10 cmH₂O.

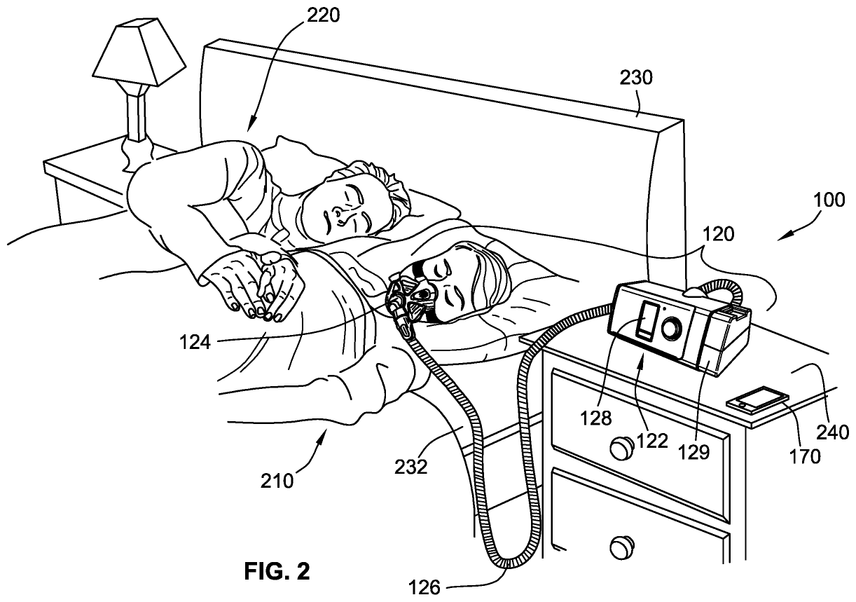

As shown in FIG. 2, in some implementations, the user interface 124 is or includes a facial mask (e.g., a full face mask) that covers the nose and mouth of the user. Alternatively, in some implementations, the user interface 124 is a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the user's teeth, a mandibular repositioning device, etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score or therapy score (also referred to as a myAir™ score, such as described in WO 2016/061629 and U.S. Patent Pub. No. 2017/0311879, each of which is hereby incorporated by reference herein in its entirety), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122. The humidification tank 129 includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory therapy device 122 or the conduit 126 can include a waterless humidifier. The waterless humidifier can incorporate sensors that interface with other sensor positioned elsewhere in system 100.

The respiratory therapy system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring generally to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (also referred to herein as a mask, e.g., a full face mask, a nasal mask, a nasal pillows mask, etc.) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, a temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178 more generally, the one or more sensors 130 can include any combination and any number of each of the sensors described and/or shown herein.

As described herein, the system 100 generally can be used to generate physiological data associated with a user (e.g., a user of the respiratory therapy system 120 shown in FIG. 2) during a sleep session. The physiological data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 122, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, flow rate data, or both. In some implementations, the physiological data generated by one or more of the sensors 130 can be used by the control system 110 to determine a sleep-wake signal associated with the user 210 during the sleep session and one or more sleep-related parameters.

The sleep-wake signal can be indicative of one or more sleep states, including sleep, wakefulness, relaxed wakefulness, micro-awakenings, or distinct sleep stages such as a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 130, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

The sleep-wake signal can also be timestamped to determine a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, an inspiration duration, and expiration duration, a number of events per hour, a pattern of events, pressure settings of the respiratory therapy device 122, or any combination thereof during the sleep session. The event(s) can include snoring, apneas, central apneas, obstructive apneas, mixed apneas, hypopneas, a mouth leak, a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, or any combination thereof. The one or more sleep-related parameters that can be determined for the user during the sleep session based on the sleep-wake signal include, for example, sleep quality metrics such as a total time in bed, a total sleep time, a sleep onset latency, a wake-after-sleep-onset parameter, a sleep efficiency, a fragmentation index, or any combination thereof.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, an inspiration duration, and expiration duration, a number of events per hour, a pattern of events, pressure settings of the respiratory therapy device 122, or any combination thereof. The event(s) can include snoring, apneas, central apneas, obstructive apneas, mixed apneas, hypopneas, a mouth leak, a mask leak (e.g., from the user interface 124), a restless leg, a sleeping disorder, choking, an increased heart rate, labored breathing, an asthma attack, an epileptic episode, a seizure, or any combination thereof.

Generally, the sleep session includes any point in time after the user 210 has laid or sat down in the bed 230 (or another area or object on which they intend to sleep), and has turned on the respiratory therapy device 122 and donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory therapy device 122, and gets out of bed 230. In some implementations, the sleep session can include additional periods of time, or can be limited to only some of the above-disclosed time periods. For example, the sleep session can be defined to encompass a period of time beginning when the respiratory therapy device 122 begins supplying the pressurized air to the airway or the user 210, ending when the respiratory therapy device 122 stops supplying the pressurized air to the airway of the user 210, and including some or all of the time points in between, when the user 210 is asleep or awake. In some implementations, the sleep session may include periods when the user is in a sleeping position either before and/or after the user starts or finishes using the respiratory therapy device.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. the user interface 124, or the conduit 126. The pressure sensor 132 can be used to determine an air pressure in the respiratory therapy device 122, an air pressure in the conduit 126, an air pressure in the user interface 124, or any combination thereof. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of a user.

The flow rate sensor 134 outputs flow rate data that can be stored in the memory device 114 and/or analyzed by the processor 112 of the control system 110. In some examples, the “flow rate data” can also be called “flow data,” whereas the “flow” is a rate of airflow measured by the flow rate sensor 134. Examples of flow rate sensors (such as, for example, the flow rate sensor 214) are described in International Publication No. WO 2012/012835 and U.S. Pat. No. 10,328,219, each of which is hereby incorporated by reference herein in its entirety. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The flow rate sensor 134 can be used to generate flow rate data associated with the user 210 (FIG. 2) of the respiratory therapy device 122 during the sleep session. Examples of flow rate sensors (such as, for example, the flow rate sensor 134) are described in International Publication No. WO 2012/012835, which is hereby incorporated by reference herein in its entirety. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user.

The temperature sensor 136 outputs temperature data that can be stored in the memory device 114 and/or analyzed by the processor 112 of the control system 110. In some implementations, the temperature sensor 136 generates temperature data indicative of a core body temperature of the user 210 (FIG. 2), a skin temperature of the user 210, a temperature of the air flowing from the respiratory therapy device 122 and/or through the conduit 126, a temperature of the air in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

The microphone 140 outputs sound data that can be stored in the memory device 114 and/or analyzed by the processor 112 of the control system 110. The microphone 140 can be used to record sound(s) during a sleep session (e.g., sounds from the user 210) to determine (e.g., using the control system 110) one or more sleep related parameters, which may include one or more events (e.g., respiratory events), as described in further detail herein. The microphone 140 can be coupled to or integrated in the respiratory therapy device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves. In one or more implementations, the sound waves can be audible to a user of the system 100 (e.g., the user 210 of FIG. 2) or inaudible to the user of the system (e.g., ultrasonic sound waves). The speaker 142 can be used, for example, as an alarm clock or to play an alert or message to the user 210 (e.g., in response to an identified body position and/or a change in body position). In some implementations, the speaker 142 can be used to communicate the audio data generated by the microphone 140 to the user. The speaker 142 can be coupled to or integrated in the respiratory therapy device 122, the user interface 124, the conduit 126, or the external device 170.

The microphone 140 and the speaker 142 can be used as separate devices. In some implementations, the microphone 140 and the speaker 142 can be combined into an acoustic sensor 141 (e.g., a SONAR sensor), as described in, for example, WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. In one or more implementations, the sound waves generated or emitted by the speaker 142 can have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters (e.g., an identified body position and/or a change in body position) described in herein such as, for example, a respiration signal, a respiration rate, an inspiration amplitude, an expiration amplitude, an inspiration-expiration ratio, a number of events per hour, a pattern of events, a sleep state, pressure settings of the respiratory therapy device 122, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned herein.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location and/or a body position of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication could be Wi-Fi, Bluetooth, or etc.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to identify a location and/or a body position of the user, to determine chest movement of the user 210, to determine air flow of the mouth and/or nose of the user 210, to determine a time when the user 210 enters the bed 230, and to determine a time when the user 210 exits the bed 230. The camera 150 can also be used to track eye movements, pupil dilation (if one or both of the user 210's eyes are open), blink rate, or any changes during REM sleep.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the user 210's breath. In some implementations, the analyte sensor 174 is positioned near the user 210's mouth to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the user 210's nose to detect analytes in breath exhaled through the user's nose. In still other implementations, the analyte sensor 174 can be positioned near the user 210's mouth when the user interface 124 is a nasal mask or a nasal pillow mask. In some implementations, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the user 210's mouth or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be positioned in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the user 210's bedroom. The moisture sensor 176 can also be used to track the user 210's biometric response to environmental changes.

One or more Light Detection and Ranging (LiDAR) sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a heart rate sensor (e.g., pulse sensor), a blood pressure sensor (e.g., sphygmomanometer sensor), an oximetry sensor, a sonar sensor, a RADAR sensor, a LiDAR sensor, a blood glucose sensor, a camera (e.g., color sensor), a pH sensor, a tilt sensor (which measures the tilt in multiple axes of a reference plane), an orientation sensor (which measures the orientation of a device relative to an orthogonal coordinate frame), or any combination thereof.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, at least one of the one or more sensors 130 is not physically and/or communicatively coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, an intentional mask leak, an unintentional mask leak, a mouth leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Non-physiological parameters can also include operational parameters of the respiratory therapy system, including flow rate, pressure, humidity of the pressurized air, speed of motor, etc. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s) such as Google Nest Hub, Google Home, Amazon Echo, Amazon Show, Alexa™-enabled devices, etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for analyzing data associated with a user's use of the respiratory therapy system 120, according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. Thus, various systems for implementing the present disclosure can be formed using any portion or portions of the components shown and described herein and/or in combination with one or more other components.

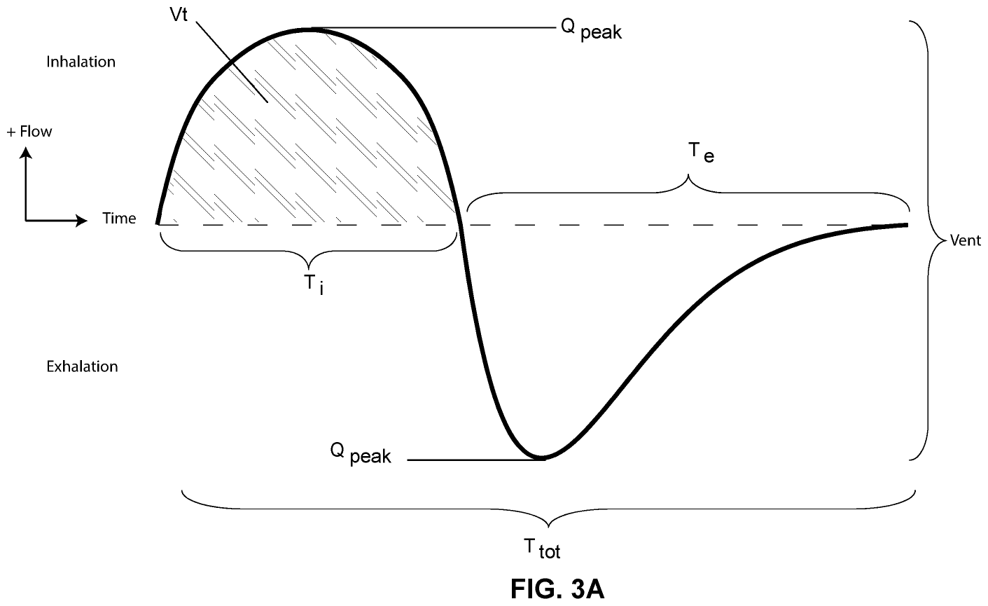

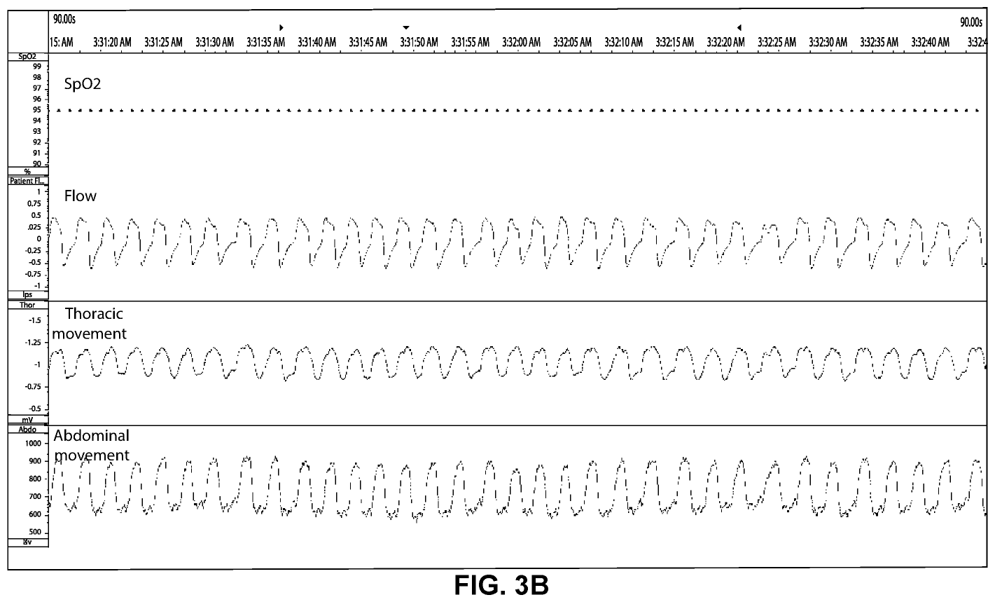

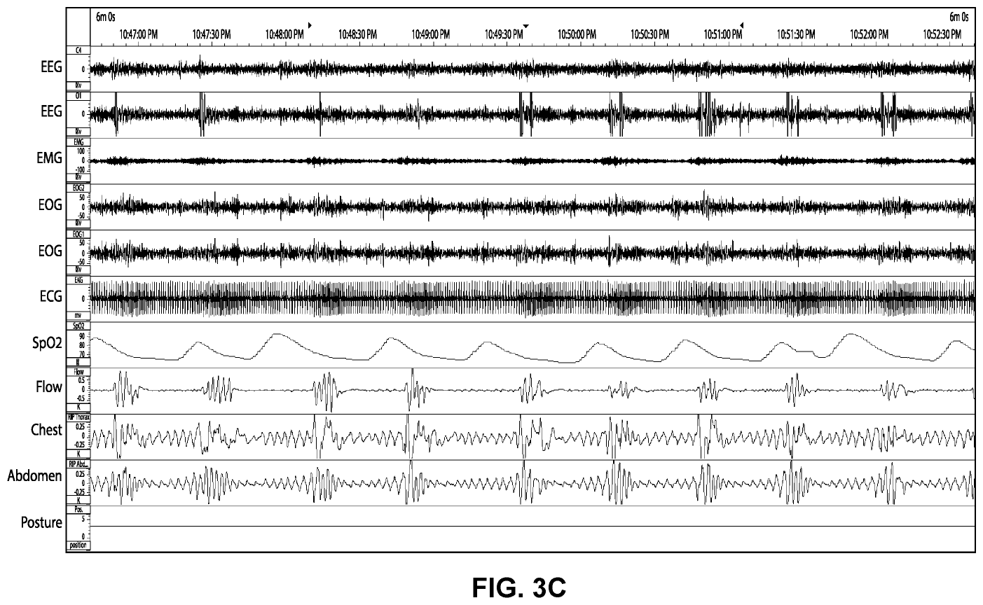

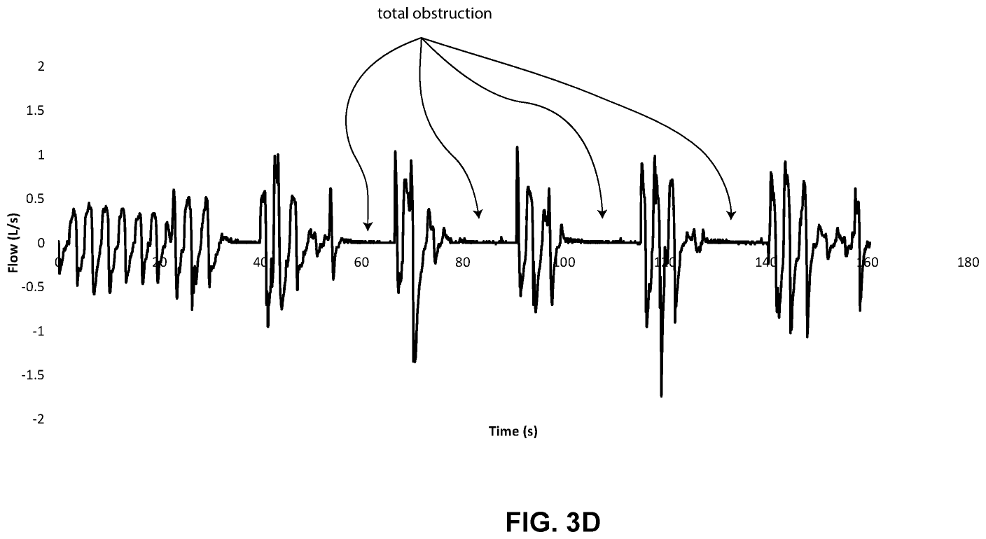

FIG. 3A illustrates a breath waveform of a person while sleeping, according to some implementations of the present disclosure. The horizontal axis is time, and the vertical axis is respiratory flow rate. While the parameter values may vary, an example breathing cycle may have the following approximate values: tidal volume Vₜ 0.5 L, inhalation time Tᵢ 1.6 s, peak inhalation flow rate Qₚₑₐₖ 0.4 L/s, exhalation time Tₑ 2.4 s, peak exhalation flow rate Qₚₑₐₖ −0.5 L/s. The total duration of the breathing cycle, Tₜₒₜ, is about four (4) seconds. An individual typically breathes at a rate of about 15 breaths per minute (BPM), with Ventilation Vent about 7.5 L/min. For an example breathing cycle, the ratio of Tᵢ to Tₜₒₜ, is about 40%.

FIG. 3B illustrates selected polysomnography channels (pulse oximetry, flow rate, thoracic movement, and abdominal movement) of an individual during non-REM sleep breathing normally over a period of about ninety seconds, with about 34 breaths, being treated with automatic PAP therapy, and the interface pressure being about 11 cmH₂O. The top channel shows pulse oximetry (oxygen saturation or SpO₂), and the scale having a range of saturation from 90 to 99% in the vertical direction. The individual maintained a saturation of about 95% throughout the period shown. The second channel shows quantitative respiratory airflow, and the scale ranges from −1 to +1 LPS in a vertical direction, and with inspiration positive. Thoracic and abdominal movement are shown in the third and fourth channels.

FIG. 3C illustrates polysomnography of an individual before treatment, according to some implementations of the present disclosure. There are eleven signal channels from top to bottom with a six-minute horizontal span in time. The top two channels are electroencephalogram (EEG) from different scalp locations. Periodic spikes in the second EEG represent cortical arousal and related activity. The third channel down is submental electromyogram (EMG). Increasing activity around the time of arousals represents genioglossus recruitment. The fourth and fifth channels are electro-oculogram (EOG). The sixth channel is an electrocardiogram (ECG). The seventh channel shows pulse oximetry (SpO₂) with repetitive desaturations to below 70% from about 90%. The eighth channel is respiratory airflow using a nasal cannula connected to a differential pressure transducer. Repetitive apneas of 25 to 35 seconds alternate with 10 to 15 second bursts of recovery breathing coinciding with EEG arousal and increased EMG activity. The ninth channel shows movement of chest and the tenth shows movement of abdomen. The abdomen shows a crescendo of movement over the length of the apnea leading to the arousal. Both become untidy during the arousal due to gross body movement during recovery hyperpnea. The apneas are therefore obstructive, and the condition is severe. The lowest channel is posture, and in this example it does not show change.

FIG. 3D illustrates flow rate data where an individual is experiencing a series of total obstructive apneas, according to some implementations of the present disclosure. The duration of the recording is approximately 160 seconds. Flow rates range from about +1 L/s to about −1.5 L/s. Each apnea lasts approximately 10-15 s.

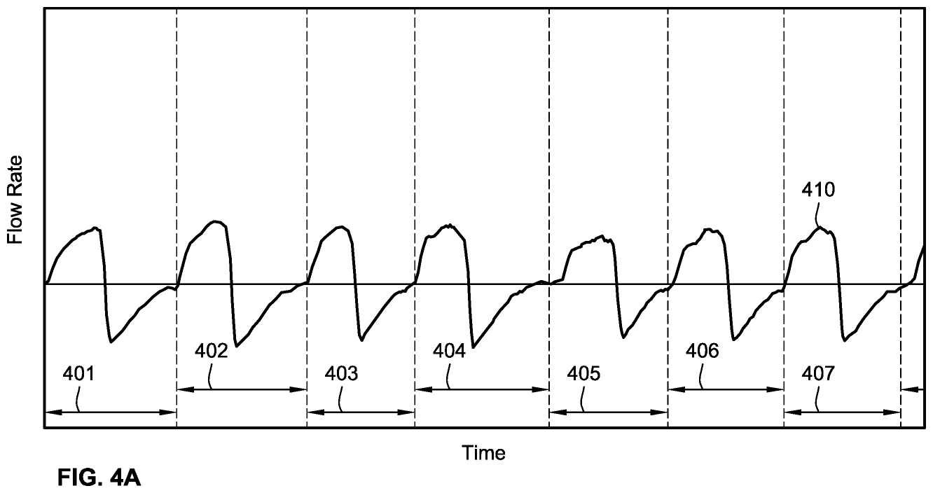

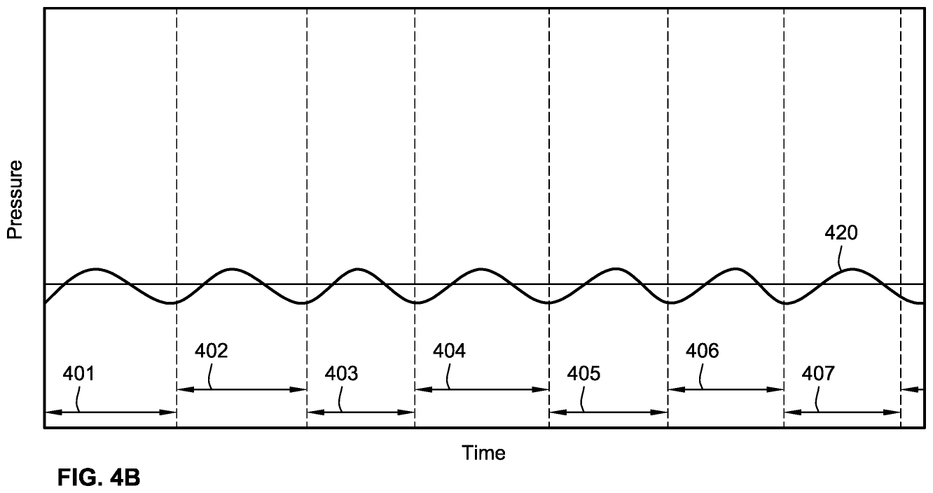

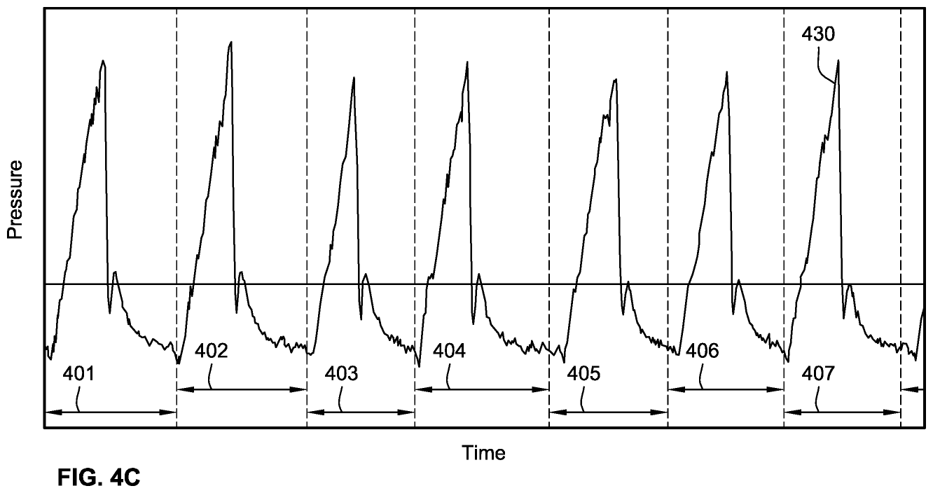

In some implementations, the system of the present disclosure includes a flow rate sensor (e.g., the flow rate sensor 134 of FIG. 1) and a pressure sensor (e.g., the pressure sensor 132 of FIG. 1). The flow rate sensor is configured to generate flow rate data over a period of therapy time. For example, FIG. 4A illustrates a portion of such flow rate data associated with a user (e.g., the user 210 of FIG. 2) of a respiratory therapy system (e.g., the respiratory therapy system 120 of FIG. 1), according to some implementations of the present disclosure. As shown in FIG. 4A, a plurality of flow rate values measured over about seven full breathing cycles (401-407) is plotted as a continuous curve 410.

In some implementations, the pressure sensor is configured to generate pressure data over a period of therapy time. For example, FIG. 4B illustrates pressure data associated with a user of a CPAP system, according to some implementations of the present disclosure. The pressure data shown in FIG. 4B was generated over the same period of therapy time as that of FIG. 4A. As shown in FIG. 4B, a plurality of pressure values measured over about seven full breathing cycles (401-407) is plotted as a continuous curve 420. Because a CPAP system is used, the continuous pressure curve of FIG. 4B exhibits a generally sinusoidal pattern with a relatively small amplitude, because the CPAP system attempts to maintain the constant predetermined air pressure for the system during the seven full breathing cycles.

Referring to FIG. 4C, pressure data associated with a user of a respiratory therapy system with an expiratory pressure relief (EPR) module is illustrated, according to some implementations of the present disclosure. The pressure data shown in FIG. 4C was generated over the same period of therapy time as that of FIG. 4A. As shown in FIG. 4C, a plurality of pressure values measured over about seven full breathing cycles (401-407) is plotted as a continuous curve 430. The continuous curve of FIG. 4C is different from that of FIG. 4B, because the EPR module (used for the pressure data in FIG. 4C) can have different settings for an EPR level, which is associated with the difference between a pressure level during inspiration and a reduced pressure level during expiration.

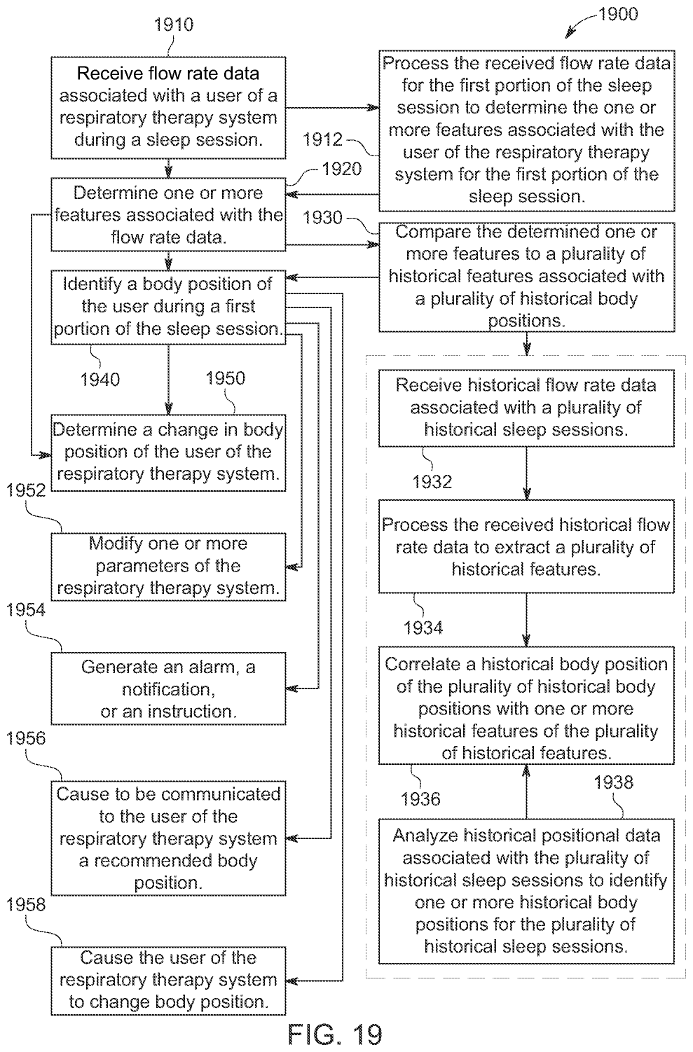

According to some implementations of the present disclosure and described in more detail herein (such as method 1900 in FIG. 19), the system 100 (FIG. 1) provides a low-cost and effective solution to determine the rotation of a user's head and/or body during their sleep using features obtained and/or derived from flow rate data, such as breath rate, amplitude changes, number of detected apnea events, noisy sections (e.g., disturbed signal) that may indicate a change of position, symbolic analysis (e.g., mathematical method) of signals. This determined rotation (e.g., body position) may be used to adapt therapy settings and/or simply present the data to the user and/or a care provider as therapy information.

In some examples, if these flow rate features are collected during the diagnostic phase with a polysomnography (PSG) system, a CPAP titration, a home sleep test (HST) night, a “fingerprint” of the user may be extracted that is indicative of the user's respiration and sleep preferences (e.g., personalized to user), and a model can then be developed for use during normal therapy. In some examples, such a model may be a set of rules (e.g., decision trees, rule-based expert systems) based on flow rate features and/or other information on the user, or a model that processes in the data pipeline of the respiratory therapy device (e.g., recurrent neural networks, echo state networks, markovian models). Further, this model may provide a degree of forecasting, hence detecting deteriorating patterns or a change of position during user's sleep, and (i) proactively change therapy settings before an apnea occurs, or (ii) reducing the intensity of therapy if the breathing remains regular.

In some implementations, changes and/or distortions in the flow rate signal may indicate that the user is moving and/or changing body position. Therefore, features obtained and/or derived from the flow rate signal (e.g., breathing rate, amplitude, variation between breaths) may be tracked to train an algorithm to learn whether the user is in a specific body position, and/or may have changed position. In some implementations, features of the flow rate signal associated with a change in the impulsiveness of the signal, spectral content of the signal, or the randomness of the signal, may be used to indicate movement of the user. For example, a relative reduction in frequency content of the signal at or around the fundamental frequency of the human respiratory rate (e.g. 6 to 30 breaths per minute, or potentially higher frequencies for children), and/or an increase in frequency content or signal amplitude and/or impulsiveness at higher frequencies may be indicative features of user movement. Analysis of the flow rate signal may include spectral analysis, such as by taking the Fourier Transform of the signal and comparing the amplitude of the signal at different frequency ranges. For example, comparing the amplitude of the signal in the range of the fundamental frequency of human respiration, with the amplitude at frequency ranges higher than the fundamental frequencies of respiration. In some implementations, the analysis can include an estimate of the amplitude modulation of the higher frequency components of the signal in the range of the fundamental frequencies of respiration. In this way, it is possible to distinguish between signal noise due to patient movement from signal noise associated with respiration, such as snoring. In some implementations, features of the flow rate signal used to estimate user movement may include any one or more, or all, of the kurtosis of the signal, the spectral entropy of the signal, variance of the signal, the variance of the envelope of the signal, the root mean square of the signal, and the variance of the root mean square of the signal. The flow rate signal may be segmented based on (i) a specified time period (e.g., three seconds, five seconds, ten seconds, 30 seconds, one minute, three minutes, five minutes, ten minutes, 15 minutes, etc.), (ii) a specified number of breaths (e.g., one breath, two breaths, three breaths, five breaths, ten breaths, 30 breaths, etc.), and/or (iii) amount of changes and/or distortions detected in the flow rate signal. In some implementations, features obtained and/or derived from the flow rate signal may be processed and/or analyzed to determine positional apnea and/or positional change.

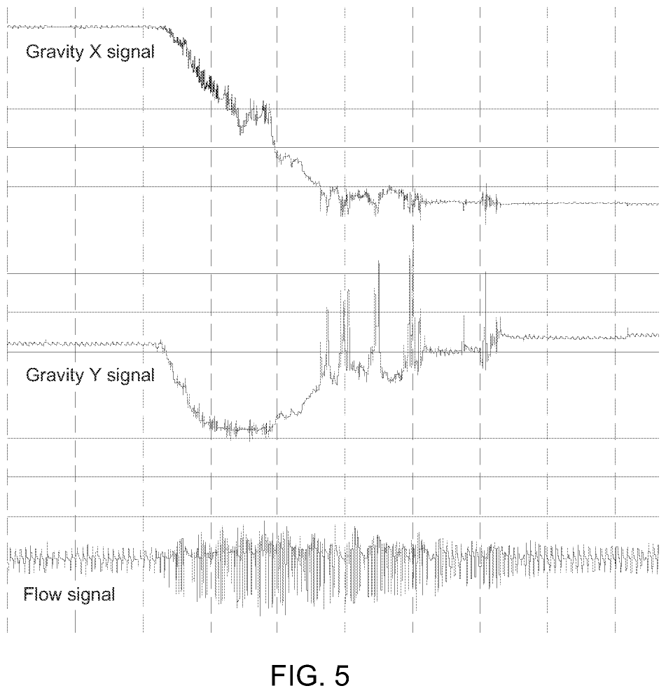

Referring to FIG. 5, example positional data and flow rate data associated with a user of a respiratory therapy system are illustrated during a position change of the user. The gravity x signal and gravity y signal obtained from a motion sensor (e.g., the motion sensor 138 of the system 100) are overlaid with the flow rate signal (e.g., obtained from the respiratory therapy device 122 of the respiratory therapy system 120). For some users, position changes during a sleep session are infrequent, thus the disclosed system for identifying body position and/or position changes does not need to run with strict real-time latencies. For example, when a change in position is detected, the system can employ the first 2-3 minutes to estimate the new position and then ramp-up/ramp-down therapeutic pressure over time. As shown in FIG. 5, changes in the gravity x signal and gravity y signal correspond with distortions in the flow rate signal. In this example, the positional data and flow rate data are segmented based on a fixed time period (shown as equally spaced vertical lines).

In some implementations, when the data segment changes, different statistical features may be extracted from the flow rate data, such as breathing rate, flow rate amplitude averages, and standard deviations, which may in turn imply a change in the position of the user. This information is useful to confirm whether the user is experiencing positional sleep apnea, which may affect the therapy parameters based on the user's body position during therapy, or may be used to recommend alternative therapies, such as positional obstructive sleep apnea therapies.

In some implementations, it may be desirable to rank or classify the user according to patterns of movement during sleep sessions, for example, it may be a sign of good sleep or good health to have relatively frequent changes of position with relatively consolidated sleep in between each position shift. In this case, it may be desirable to notify the user or a third party that by this criterion the sleep or health of the user appears to be good or normal. Alternatively, it may be an indication of poor sleep or health to have very frequent shifts in position. Likewise, it may be a sign of poor sleep or poor health to have very few shifts in position. In some of cases, it may be desirable to notify the user or third party that the frequency of position shifts may be indicative of an issue. In some implementations, the number or rate of position shifts may be used to predict or screen for other conditions, such as heart failure, risk of stroke, obesity, lung disease, or merely as a predictor of the user's age or health condition. In some implementations, it is desirable to estimate a baseline expected rate of position shifts based on demographic information, such as age, and/or other conditions, such as a particular disease state, such as heart failure, and to evaluate the individual user in reference to the baseline in order to determine a risk of an additional condition, such as, for example, a cardiac arrhythmia. In some implementations, it may be desirable to use the number of position shifts during sleep to estimate the age range or general health of the user. In some implementations, it is desirable to guide the user to seek further diagnosis or treatment, such as for heart failure, cardiac arrhythmia, insomnia, and/or respiratory conditions.

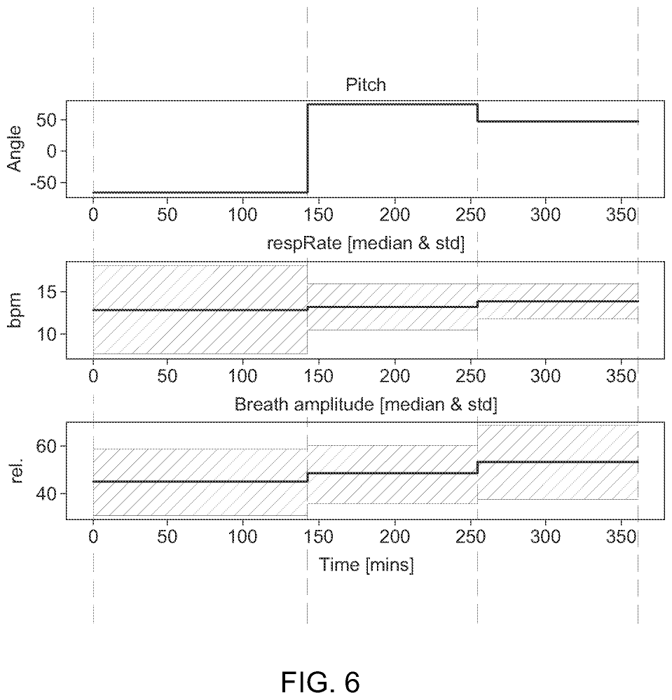

FIG. 6 illustrates example corresponding changes in respiratory patterns (e.g., shown as features derived from the flow rate signal) after a positional change of a user of a respiratory therapy system. The “pitch” plot illustrates the time in minutes as the x-axis, and the body angle in degrees as the y-axis. The angle may be estimated from accelerometer data obtained from a motion sensor (e.g., the motion sensor 138 of the system 100), where the gravity x signal and the gravity y signal are converted into the angle. In some examples, zero (or about zero) corresponds to the supine body position, thus any other body position creates a non-zero angle (e.g., −90° or about −90° is perfectly on the left side; and +90° or about +90° is perfectly on the right side). In some examples, the angle is a value between −180° to +180°. The angle being a positive value is associated with right lateral body position, the angle being a negative value is associated with left lateral body position, the angle being zero is associated with the supine body position, and the angle being +180 (or about +180) degrees or −180 (or about −180) degrees is associated with prone body position. As shown in FIG. 6, zero at the angle axis corresponds to the supine body position, −50° (or about −50°) is on the left, and +50° (or about +50°) is on the right.

The “respRate” plot illustrates the time in minutes as the x-axis, and breaths per minute as the y-axis. For each position determined by the “pitch” plot, the median and the standard deviation of the respiratory rates are illustrated in the “respRate” plot. The standard deviation can be indicative of whether the user's respiratory rate during a specific body position is erratic. In this example, every time the user moves to a new body position, the median respiratory rate increases. However, the respiratory rate for the user is more erratic when the user is on the left side than on the right side.

The “breath amplitude” plot illustrates the time in minutes as the x-axis, and the relative flow rate amplitude as the y-axis. For each position determined by the “pitch” plot, the median and the standard deviation of the relative flow rate amplitudes are illustrated in the “breath amplitude” plot. In this example, every time the user moves to a new body position, the median relative flow rate amplitude also increases, with each level corresponding to a different body position. In some implementations, a regular pattern in breathing amplitude (e.g., represented as relative flow rate amplitude) may indicate restorative sleeping phases, while an erratic breathing amplitude may indicate the presence of restricted air flow, apneas, and/or a suboptimal body position during sleep for a user.

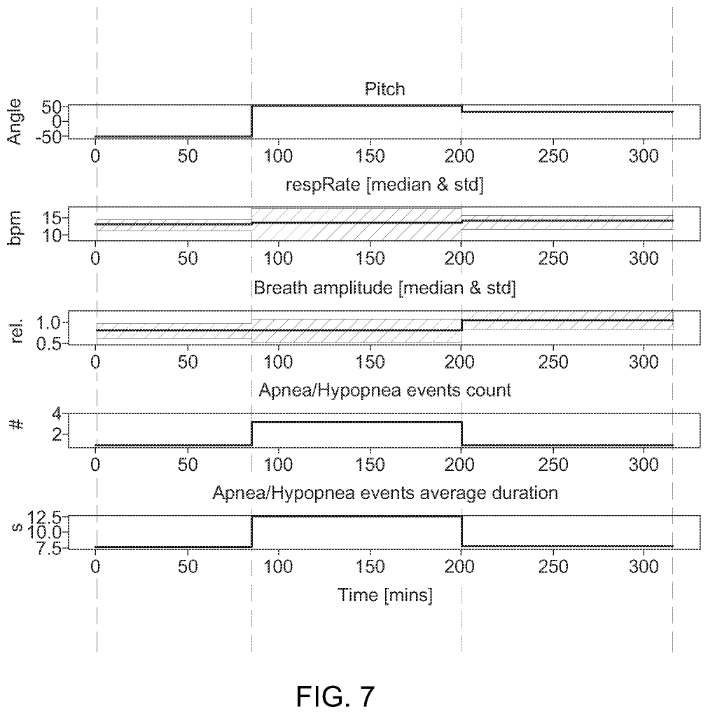

FIG. 7 illustrates example corresponding changes in respiratory patterns (e.g., shown as features derived from the flow rate signal) after a positional change, and associated apnea/hypopnea data of a user of a respiratory therapy system. Similar to FIG. 6, the “pitch” plot illustrates the time in minutes as the x-axis, and the angle in degrees as the y-axis. The “respRate” plot illustrates the time in minutes as the x-axis, and breaths per minute as the y-axis. For each position determined by the “pitch” plot, the median and the standard deviation of the respiratory rates are illustrated in the “respRate” plot. In this example, every time the user moves to a new body position, the median respiratory rate increases. However, after moving from the left side to the right side body position (from approximately −45 to approximately +65), the respiratory rate for the user is more erratic than staying in either the left side or the right side without much movement.

The “breath amplitude” plot illustrates the time in minutes as the x-axis, and the relative flow rate amplitude as the y-axis. For each position determined by the “pitch” plot, the median and the standard deviation of the relative flow rate amplitudes are illustrated in the “breath amplitude” plot. In this example, the median relative flow rate amplitude does not always increase in response to a change in body position. However, for this user, the relative flow rate amplitude is more erratic than staying in either the left side or the right side without much movement.

Additionally or alternatively, in some implementations, the detection of AHI may be indicative of a specific body position relative to a user. The “Apnea/Hypopnea events count” plot illustrates the time in minutes as the x-axis, and number of events as the y-axis. The “Apnea/Hypopnea events average duration” plot illustrates the time in minutes as the x-axis, and average duration in seconds as the y-axis. For this user, in the central segment, the number of events increases, which is reflected in the standard deviation of the respiratory rate. Thus, it is likely that this user has positional apnea, which is more severe when the user switches from the left side to the right side body position.

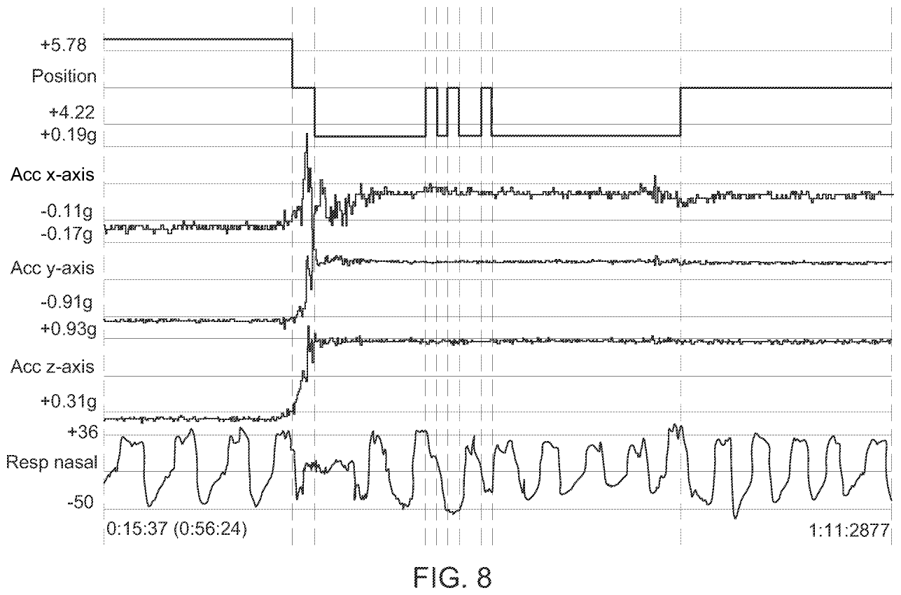

FIGS. 8-10 illustrate example positional data and flow rate data associated with three different users of their corresponding respiratory therapy systems. The “Position” line represents the angle calculated from the data obtained from a three-axis accelerometer (shown as “Acc x-axis,” “Acc y-axis,” “Acc z-axis”), where a positive value indicates the user is on the right side, a negative value indicates the user is on the left side, and a zero indicates the user being in the supine body position. The “Resp nasal” line represents the flow rate signal generated by the corresponding respiratory therapy system for each user.

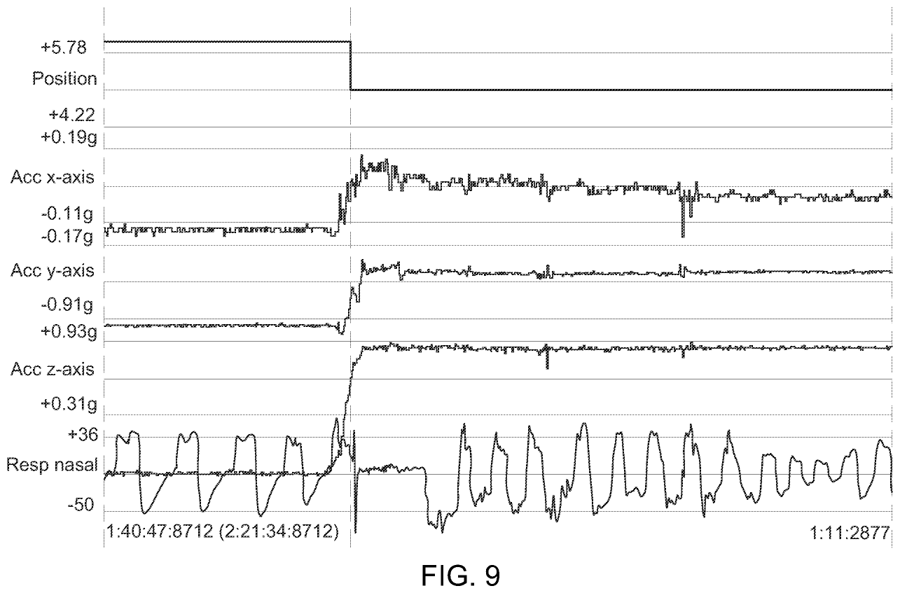

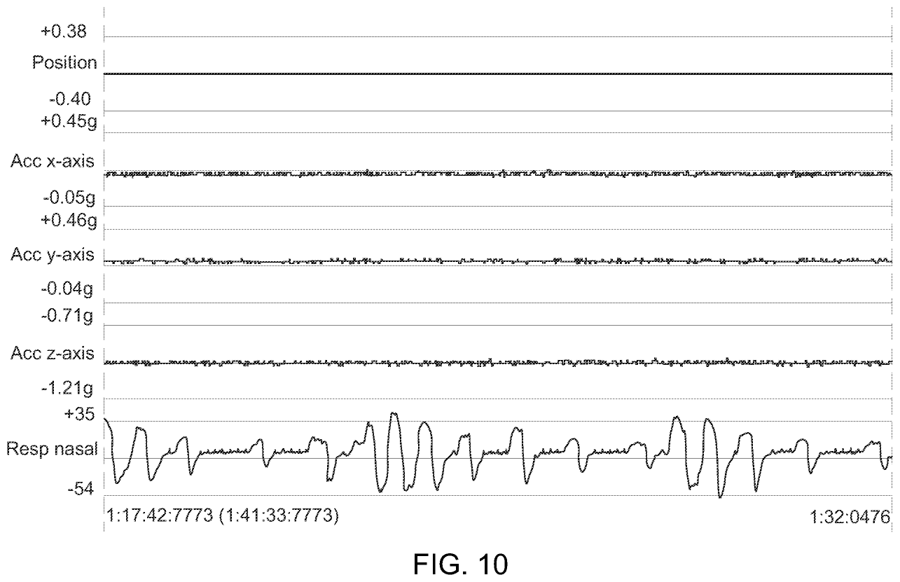

As shown in FIG. 8, the more drastic movement from the right side to the left side (stopping briefly in the supine body position) corresponds to resultant irregularities in the flow rate signal. As shown in FIG. 9, the movement from the right side to the supine body position also corresponds to resultant irregularities in the flow rate signal. Referring to FIG. 10, the user did not move, but was experiencing hypopneas, which is reflected in the flow rate signal (see e.g. the “flat” sections of the “Resp nasal” signal). Thus, according to some implementations of the present disclosure, the flow rate signal can be analyzed to differentiate effects from events related to a sleep disorder and events related to body positions.

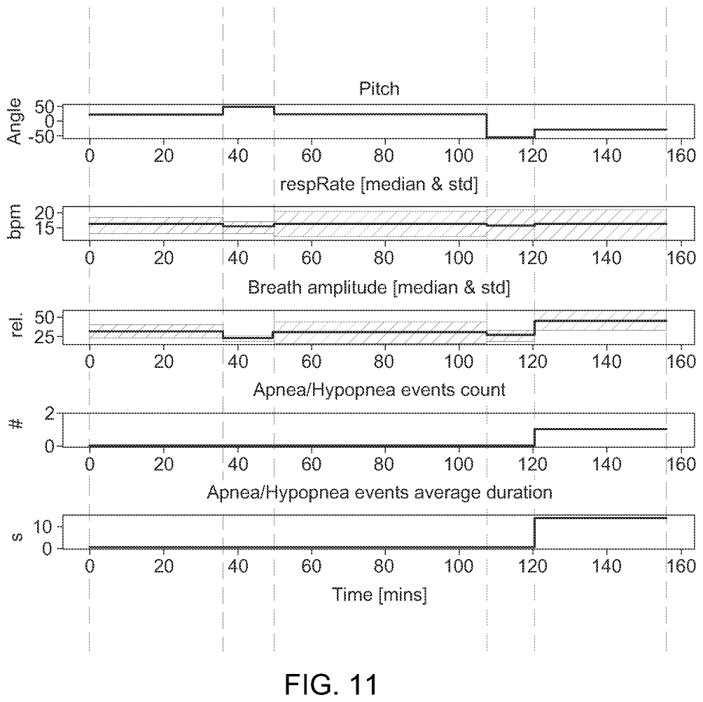

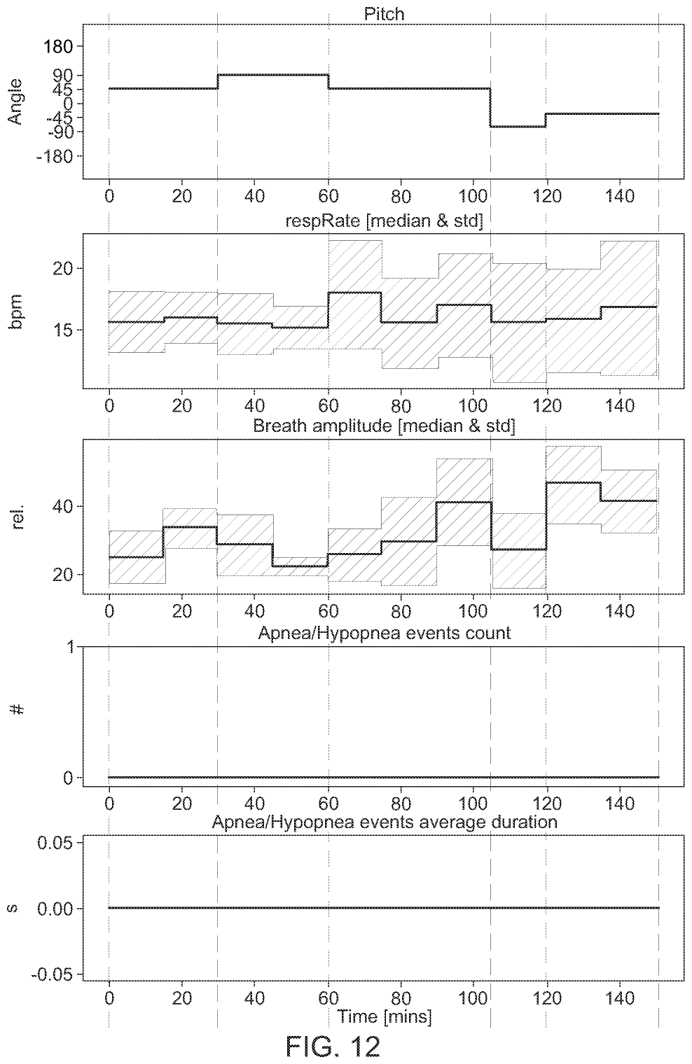

As described herein, there are different methods to segment the flow rate data to extract and/or determine features for correlating the features with the body positions. In some implementations, the flow rate data may be analyzed for each determined body position, such as shown in FIGS. 11, 13, 15, 17. Additionally or alternatively, in some implementations, the flow rate data may be analyzed for every specified time period, such as shown in FIGS. 12, 14, 16, 18.

The five plots in each of FIGS. 11-18 illustrate the parameters that are the same, or similar to, those illustrated in FIG. 7. FIG. 11 illustrates analyzed positional data, features derived from flow rate data, and apnea/hypopnea data associated with a user of a respiratory therapy system, segmented by each determined angle. FIG. 12 illustrates analyzed positional data, features derived from flow rate data, and apnea/hypopnea data associated with the user of FIG. 11, segmented by 15 minutes. The raw data in FIGS. 11-12 was the same, except that the segmentation methods are different.

Referring generally to FIGS. 11-12, the user stays mostly on the right side (e.g., from minute 0 to approximately minute 108), then switches to the left side (e.g., from approximately minute 108 to approximately minute 156). There are some slight movement (e.g., at approximately minute 38 and at approximately minute 50) while the user is on the left side, and some slight movement (e.g., at approximately minute 120) while the user is on the right side.

As shown in FIG. 11, the pitch of the user is plotted, with time (in minutes) as the X-axis, and angle (in degrees) as the Y-axis. The median and the standard deviation of the corresponding respiratory rate of the user are plotted, with time (in minutes) as the X-axis, and breaths per minute (bpm) as the Y-axis. The median and the standard deviation of the corresponding breath amplitude are plotted, with time (in minutes) as the X-axis, and a relative value as the Y-axis. The apnea/hypopnea events count is plotted, with time (in minutes) as the X-axis, and the number of apnea or hypopnea events as the Y-axis. The apnea/hypopnea events average duration is plotted, with time (in minutes) as the X-axis, and the duration of apnea or hypopnea events (in seconds) as the Y-axis. For this user, each change in body position corresponds to a change (e.g., increase or decrease) in median respiratory rate, and a change (e.g., increase or decrease) in median relative flow rate amplitude. The respiratory rate is more erratic leading up to an apnea/hypopnea event. The user experiences apnea/hypopnea while on the left side, but not on the right side, which is also reflected in the median relative flow rate amplitude.

As shown in FIG. 12, while the raw data is the same as that in FIG. 11, because the segmentation methods are different, no apnea/hypopnea events are detected in FIG. 12. Segmenting by every 15 minutes, FIG. 12 shows that both the respiratory rate and the relative flow rate amplitude are more erratic leading up to (e.g. approximately 45 minutes prior), during, and after the user changes from sleeping on the right side to the left side. In addition, the relative flow rate amplitude increases when the user stays on the left side, which suggests that the user breathes more heavily in the left side body position.

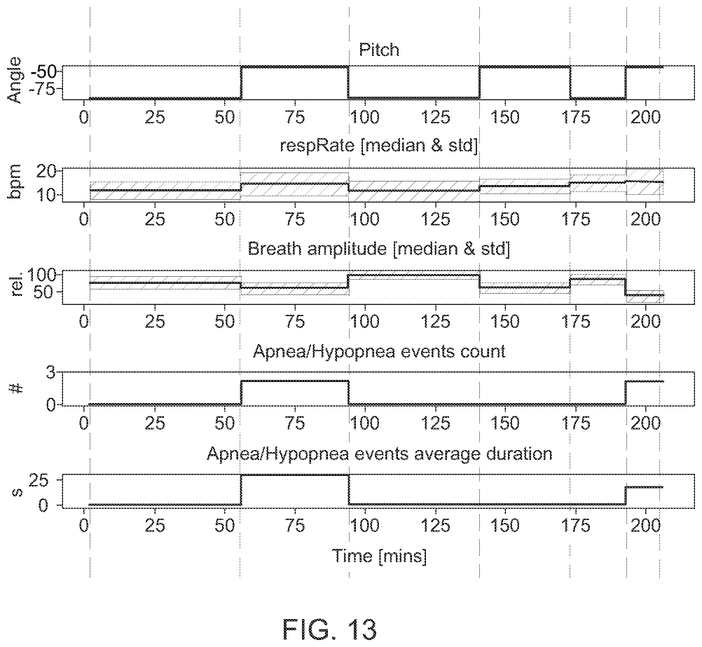

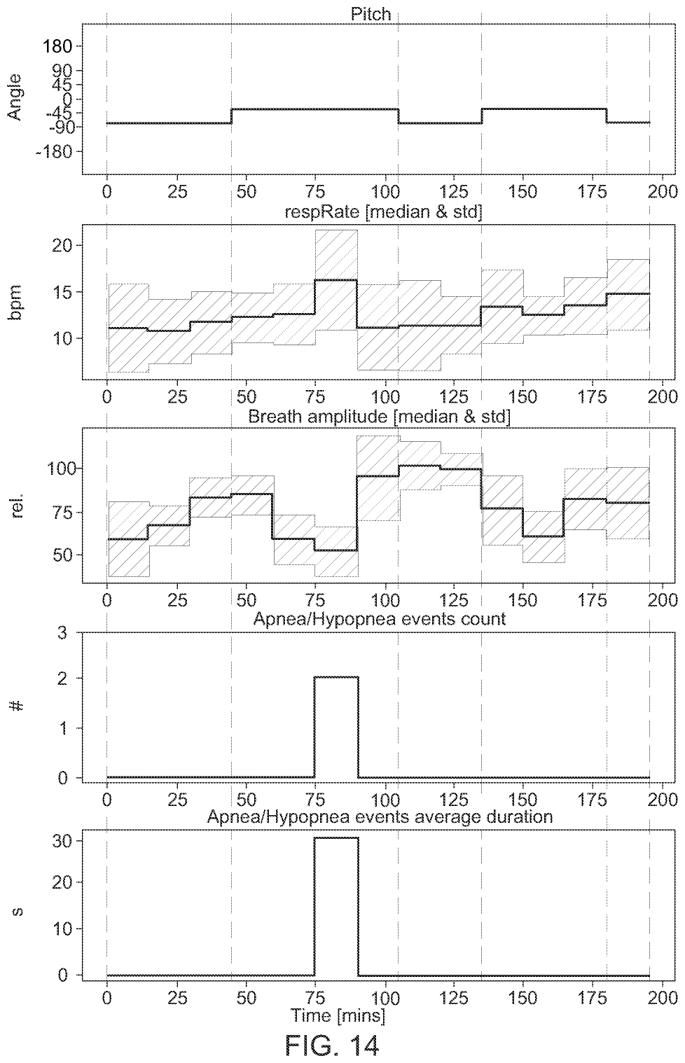

FIG. 13 illustrates analyzed positional data, features derived from flow rate data, and apnea/hypopnea data associated with a user of a respiratory therapy system, segmented by each determined angle. FIG. 14 illustrates analyzed positional data, features derived from flow rate data, and apnea/hypopnea data associated with the user of FIG. 13, segmented by 15 minutes. The raw data in FIGS. 13-14 was the same, except that the segmentation methods are different.

Referring generally to FIGS. 13-14, the user sleeps on the left side for the entire duration shown, but moves back and forth between approximately 90 degrees to approximately 45 degrees. As shown in FIG. 13, for this user, each change in body position corresponds to a change (e.g., increase or decrease) in median respiratory rate, and a change (e.g., increase or decrease) in median relative flow rate amplitude. The respiratory rate is more erratic while the user experiences an apnea/hypopnea event. As shown in FIG. 14, while the raw data is the same as that in FIG. 13, because the segmentation methods are different, FIG. 14 shows that the respiratory rate is more erratic while the user experiences an apnea/hypopnea event, but not the relative flow rate amplitude.

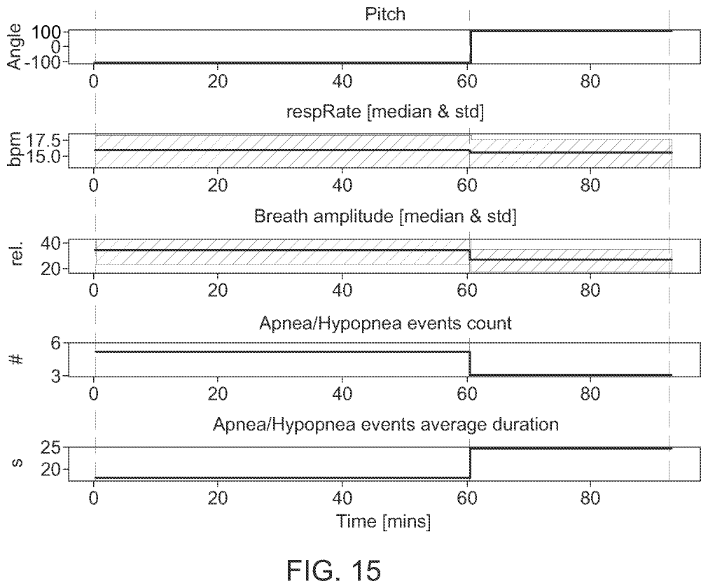

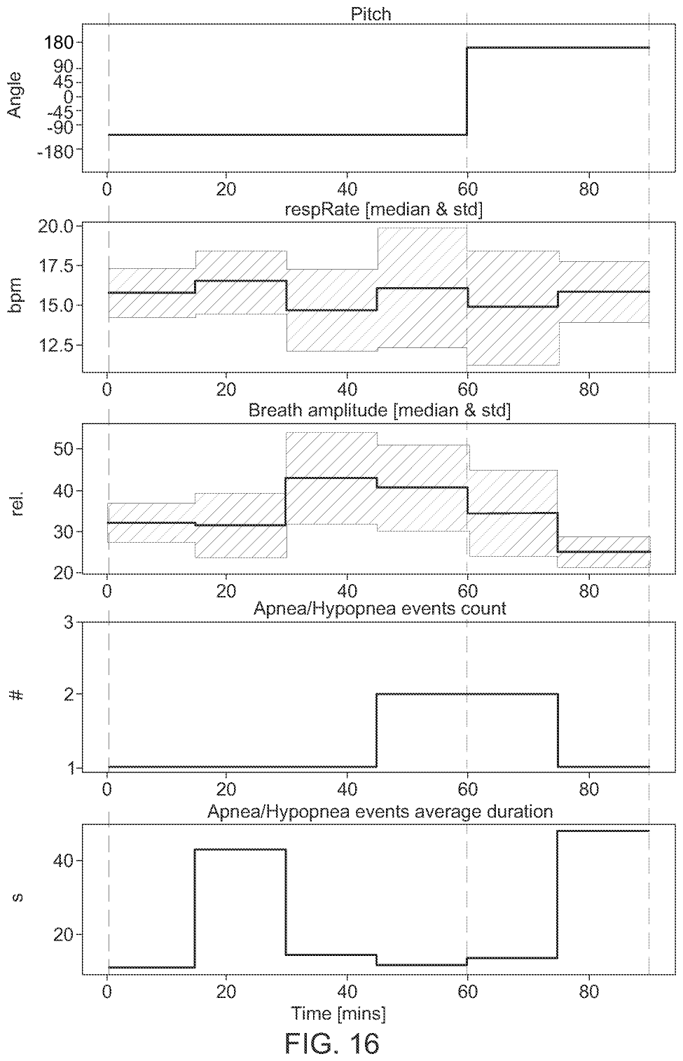

FIG. 15 illustrates analyzed positional data, features derived from flow rate data, and apnea/hypopnea data associated with a user of a respiratory therapy system, segmented by each determined angle. FIG. 16 illustrates analyzed positional data, features derived from flow rate data, and apnea/hypopnea data associated with the user of FIG. 15, segmented by 15 minutes. The raw data in FIGS. 15-16 was the same, except that the segmentation methods are different.

In some implementations, the apnea/hypopnea events count and the duration of apnea/hypopnea events can be derived and/or determined from (i) the respiratory rate, (ii) the breath amplitude, or (iii) both (i) and (ii). Thus, in some such implementations, only the respiratory rate and/or the breath amplitude is needed to correlate to an angle of the user, thereby identifying the body position of the user. In some examples, both the respiratory rate and the breath amplitude are derived and/or determined from the airflow data, such as the flow rate data, the pressure data, or both.

In some implementations, the apnea/hypopnea events count and the duration of apnea/hypopnea events are not necessary to determine the body position of the user (e.g., in healthy users, in treated users that do not experience apnea/hypopnea events in a particular time period, or in users that experience apnea/hypopnea events). In some such implementations, the system (e.g., a trained machine learning model) can identify the body position of the user with only the respiratory rate (median, standard deviation, or both) and/or the breath amplitude (median, standard deviation, or both).

In some implementations, the respiratory pressure therapy level may be automatically adjusted to treat events associated with upper airway flow resistance, such as apneas, hypopneas, inspiratory flow limitation, or snore, such that variation in the threshold level, below which particular events occurring may be predictive of positional OSA. In some implementations, the respiratory therapy device may detect a suspected change in user position, and respond by waiting a predetermined length of time, such as five minutes, or a dynamically determined period of time, such as until a respiration pattern has been established, and then gradually lowering the therapy pressure to determine the threshold pressure below which respiratory-related events occur. Upon determining the threshold value, the therapy mode may revert to its normal operation.

Referring generally to FIGS. 15-16, the user stays on the left side (e.g., from minute 0 to approximately minute 60), then switches to the prone body position (e.g., from approximately minute 60 to approximately minute 90), with little movement otherwise. As shown in FIG. 15, for this user, the change in body position corresponds to a change (e.g., a decrease) in median respiratory rate, and a change (e.g., a decrease) in median relative flow rate amplitude. The user experiences apnea/hypopnea in both body positions, but the events last longer on average when the user is in the prone body position.

As shown in FIG. 16, while the raw data is the same as that in FIG. 15, because the segmentation methods are different, FIG. 16 shows that the respiratory rate is more erratic while the user experiences more frequent apnea/hypopnea events, whereas the relative flow rate amplitude is more erratic leading up to and while the user experiences more frequent apnea/hypopnea events.

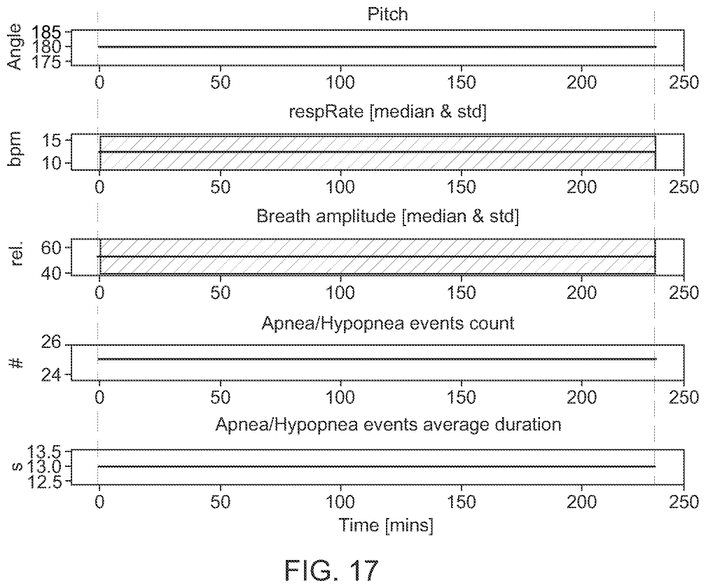

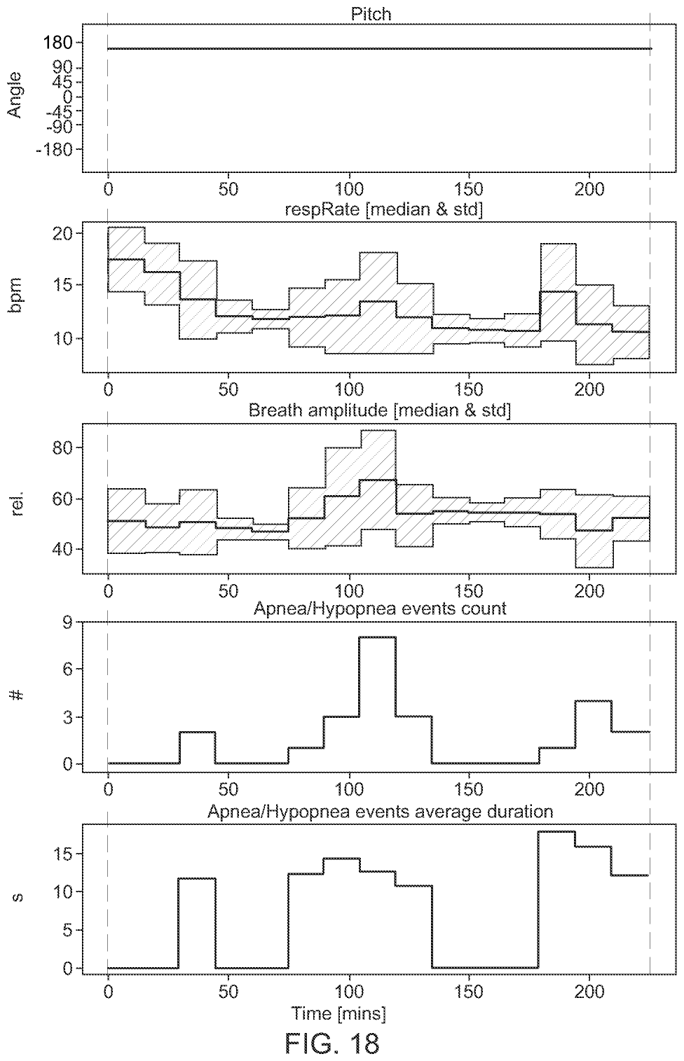

FIG. 17 illustrates analyzed positional data, features derived from flow rate data, and apnea/hypopnea data associated with a user of a respiratory therapy system, segmented by each determined angle. FIG. 18 illustrates analyzed positional data, features derived from flow rate data, and apnea/hypopnea data associated with the user of FIG. 18, segmented by 15 minutes. The raw data in FIGS. 17-18 was the same, except that the segmentation methods are different.

The user in FIGS. 17-18 did not wear the motion sensor (e.g., the three-axis accelerometer), thus FIG. 17 shows no change in any of the plots. However, because FIG. 18 is segmented by 15 minutes, the median value and standard deviation for each segment vary for the respiratory rate and the flow rate amplitude. FIG. 18 also shows the apnea/hypopnea event counts and the average duration vary over time. In this example and others, the features extracted and/or determined from the flow rate signal can be analyzed to identify the body position and/or change in body position of the user, while taking into account effects from apnea/hypopnea events.

Referring to FIG. 19, a flow diagram for a method 1900 for identifying a body position of a user of a respiratory therapy system during a sleep session is disclosed. One or more steps of the method 1900 can be implemented using any element or aspect of the system 100 (FIGS. 1-2) described herein. The method 1900 for identifying body position while on therapy is advantageous because based on data collected from the user, provide proactive actions may be provided during therapy to ensure both effective and comfortable therapy settings. In addition, a cumbersome external device (e.g. undershirt bumpers, chest straps) is not needed in order to provide positional therapy.

At step 1910, airflow data (e.g., flow rate data, pressure data, or both) associated with a user of a respiratory therapy system during a sleep session is received. At step 1920, one or more features associated with the airflow data is determined. In some implementations, the one or more features are derived and/or calculated from the raw flow signal (or flow rate signal). In some implementations, the one or more features determined at step 1920 includes a respiratory rate, a change in respiratory rate, an amplitude of flow rate signal (“breath amplitude”), a change in amplitude of the flow rate signal (“change in breath amplitude”), a number of apnea events, a number of hypopnea events, a duration of apnea events, a duration of hypopnea events, or any combination thereof. In some such implementations, the determined one or more features are derived from the airflow data. In some such implementations, the airflow data is flow rate data determined from the flow rate signal.

For example, in some implementations, the determined one or more features include a respiratory rate, an amplitude of flow rate signal, or both. In some such implementations, the determined one or more features further include a number of apnea or hypopnea events, a duration of apnea or hypopnea events, or both.

As another example, in some implementations, the determined one or more features include a respiration rate, a change in respiration rate, or both. In some implementations, the determined one or more features include an amplitude of flow rate signal, a change in amplitude of the flow rate signal, or both. Thus, in some implementations, the determined one or more features include a respiratory rate and an amplitude of flow rate signal. In some implementations, the determined one or more features include a respiratory rate and a change in amplitude of flow rate signal. In some implementations, the determined one or more features include a change in respiratory rate and an amplitude of flow rate signal. In some implementations, the determined one or more features include a change in respiratory rate and a change in amplitude of flow rate signal. In some implementations, the determined one or more features include, or further include, a number of apnea or hypopnea events, a duration of apnea or hypopnea events, or both. Thus, in some implementations, the determined one or more features include a respiratory rate, a number of apnea or hypopnea events, or both. In some implementations, the determined one or more features include a respiratory rate, a duration of apnea or hypopnea events, or both. In some implementations, the determined one or more features include an amplitude of flow rate signal, a number of apnea or hypopnea events, or both. In some implementations, the determined one or more features include an amplitude of the flow rate signal, a duration of apnea or hypopnea events, or both. In some implementations, the determined one or more features include a change in respiratory rate, a number of apnea or hypopnea events, or both. In some implementations, the determined one or more features include a change in respiratory rate, a duration of apnea or hypopnea events, or both. In some implementations, the determined one or more features include a change in amplitude of flow rate signal, a number of apnea or hypopnea events, or both. In some implementations, the determined one or more features include a change in amplitude of the flow rate signal, a duration of apnea or hypopnea events, or both.

In some implementations, at step 1912, the airflow data received at step 1910 is processed for a first portion of the sleep session to determine the one or more features associated with the user of the respiratory therapy system for the first portion of the sleep session. In some examples, the first portion of the sleep session is two seconds, three seconds, four seconds, five seconds, six seconds, seven seconds, eight seconds, nine seconds, ten seconds, or longer. In some examples, the first portion of the sleep session corresponds to a number of breaths, such as one breath, two breaths, three breaths, four breaths, or five breaths.

In some implementations, the one or more features determined at step 1920 includes a median respiratory rate for the first portion of the sleep session, a standard deviation in respiratory rate for the first portion of the sleep session, a median amplitude of flow rate signal for the first portion of the sleep session, a standard deviation in amplitude of the flow rate signal for the first portion of the sleep session, a mean therapy pressure for the first portion of the sleep session, or any combination thereof.

For example, in some implementations, the determined one or more features include, or further include, a median respiratory rate for the first portion of the sleep session, a standard deviation in respiratory rate for the first portion of the sleep session, or both. In some such implementations, the determined one or more features may further include (i) a median amplitude of flow rate signal for the first portion of the sleep session, (ii) a standard deviation in amplitude of the flow rate signal for the first portion of the sleep session, or (iii) both (i) and (ii).

As another example, in some implementations, the determined one or more features include, or further include, a median respiratory rate for the first portion of the sleep session, a median amplitude of flow rate signal for the first portion of the sleep session, or both. In some such implementations, the determined one or more features may further include a standard deviation in amplitude of the flow rate signal for the first portion of the sleep session.

As a further example, in some implementations, the determined one or more features include, or further include, a median respiratory rate for the first portion of the sleep session, a standard deviation in amplitude of the flow rate signal for the first portion of the sleep session, or both. In some implementations, the determined one or more features include, or further include, a standard deviation in respiratory rate for the first portion of the sleep session, a median amplitude of flow rate signal for the first portion of the sleep session, or both. In some implementations, the determined one or more features include, or further include, a standard deviation in respiratory rate for the first portion of the sleep session, a standard deviation in amplitude of the flow rate signal for the first portion of the sleep session, or both. In some implementations, the determined one or more features include, or further include, a median amplitude of flow rate signal for the first portion of the sleep session, a standard deviation in amplitude of the flow rate signal for the first portion of the sleep session, or both.

In some implementations, at step 1930, the one or more features determined at step 1920 is compared to a plurality of historical features associated with a plurality of historical body positions. The plurality of historical features and the plurality of historical body positions may be obtained from stored data (e.g., training data, objectively and/or subjectively collected data, etc.) associated with (i) the user of the respiratory therapy system, (ii) one or more other users of one or more other respiratory therapy systems, or (iii) both (i) and (ii).

In some implementations, the plurality of historical features at step 1930 is the same as the one or more features determined at step 1920 for comparison purposes, although they can be measured by the same or different sensors. For example, the plurality of historical features at step 1930 may be derived from data measured by a home sleep test, whereas the one or more features determined at step 1920 may be derived from data measured by a PAP system.

The plurality of historical features was associated with the plurality of historical body positions by one or more steps of 1932, 1934, 1936, and 1938. At step 1932, historical airflow data (e.g., historical flow rate data, historical pressure data, or both) associated with a plurality of historical sleep sessions (e.g., at least 5, at least 10, at least 50, at least 100, at least 500, at least 1000, etc.) is received. At step 1934, the historical airflow data received at step 1932 is processed to extract a plurality of historical features. At step 1936, a historical body position of the plurality of historical body positions is correlated with one or more historical features of the plurality of historical features extracted at step 1934.

Additionally or alternatively, in some implementations, at step 1938, historical positional data associated with the plurality of historical sleep sessions is analyzed to identify one or more historical body positions for the plurality of historical sleep sessions. The historical positional data is received from a contact motion sensor, a non-contact motion sensor, or both. The contact motion sensor can include an accelerometer (e.g., a two-axis accelerometer, a three-axis accelerometer), a gyroscope, a magnetometer, or any combination thereof. The non-contact motion sensor can include a camera, a mobile device, a sonar sensor, a RADAR sensor, a LiDAR sensor, atilt sensor, an orientation sensor, or any combination thereof.

In some implementations, the historical positional data includes accelerometer data. Thus, the historical positional data analyzed at step 1938 may include processing the accelerometer data to determine an angle (e.g., as shown in FIGS. 6-18). The determined angle may be associated with a degree relative to a predetermined body position of the user. In some such implementations, the predetermined body position is the supine body position. In some examples, the angle being a positive value is associated with right lateral body position, the angle being a negative value is associated with left lateral body position, the angle being zero is associated with the supine body position, and the angle being +180 (or about +180) degrees or −180 (or about −180) degrees is associated with prone body position.

At step 1940, a body position of the user during the first portion of the sleep session is identified, based at least in part on the one or more features determined at step 1920. In some implementations, the presence and/or magnitude of a relationship between the body position and the severity of sleep-disordered breathing is established. In some implementations, the identifying the body position of the user includes correlating the determined one or more features with an angle associated with the user. For example, in some such implementations, the angle is associated with a degree relative to a supine body position of the user. That is, in this example, correlating the determined one or more features with the angle does not require reference to historical data (e.g., historical data as shown in steps 1932-1938). In some such implementations, the features are fed into a trained machine learning model. The historical data may have been used to train the machine learning model, but no longer necessarily play a part in the classification and/or correlation of the airflow data features with an angle.

In some implementations, the body position may be estimated as any number of positions that are classified according to their belonging to one of two classes, where a first class includes any position that increases the likelihood of sleep disordered breathing compared to any other group of positions, such that any other position would consequently fall into the second class, defined as any position where the likelihood of the user experiencing sleep disordered breathing is less than it would be for the first class. In the case where more than two distinct likelihoods are estimated, the classification model can be extended to incorporate a higher number of classes. In some implementations, the classification model can be restricted to two classes, which are separated by a threshold value for an estimate of the likelihood of the user experiencing sleep disordered breathing. In some implementations the positions falling into the classification system can include any body positions, inclusive of all permutations of orientation of the various body parts, for example, the head, and the torso. Accordingly, in some implementations, useful information is extracted without necessarily determining the actual position of the user, but rather by determining if, and/or to what degree varying positions influence the likelihood of sleep disordered breathing.

In some implementations, the body position at step 1940 is identified based at least in part on the comparison at step 1930. As described herein, the body position of the user of the respiratory therapy system can include: generally supine, generally left lateral, generally right lateral, or generally prone. Additionally or alternatively, the body position of the user of the respiratory therapy system can be a degree relative to supine. Further additionally or alternatively, the body position of the user of the respiratory therapy system can be generally horizontal relative to ground or a degree from horizontal relative to ground.

In some implementations, at step 1950, a change in body position of the user of the respiratory therapy system is determined based at least in part on (i) the one or more features determined at step 1920, (ii) the body position of the user for the first portion of the sleep session identified at step 1940, or (iii) both (i) and (ii). For example, in some implementations, based at least in part on the one or more features determined at step 1920, the body position of the user of the respiratory therapy system is identified for a second portion of the sleep session immediately following the first portion of the sleep session. In this example, the change in body position of the user is determined, at step 1950, based at least in part on comparing the body position of the user for the first portion of the sleep session and the body position of the user for the second portion of the sleep session.

Additionally or alternatively, in some implementations, based at least in part on the one or more features determined at step 1920, the body position of the user of the respiratory therapy system is identified for a third portion of the sleep session, which is not immediately after the first portion of the sleep session. In some such implementations, there may be one or more intervening portions between the first portion and the third portion of the sleep session such as the second portion of the sleep session described herein. In some such implementations, there may be one or more intervening body positions or changes in body positions between the first portion and the third portion of the sleep session.

Additionally or alternatively, in some implementations, the change in body position of the user of the respiratory therapy system is determined, at step 1950, based at least in part on a change in value of the one or more features determined at step 1920. For example, the change in value of the one or more features determined at step 1920 can be compared to a threshold value for those one or more features.

Based at least in part on the body position of the user identified at step 1940, an action is caused to be performed. In some implementations, the action is caused to be performed during (i) a first portion of the sleep session, (ii) a subsequent portion of the sleep session (e.g., the second and/or third portions of the sleep session), or (ii) a subsequent (e.g., next) sleep session. For example, in some implementations, at step 1952, one or more parameters of the respiratory therapy system are modified based at least in part on the body position of the user identified at step 1940, such as during the first portion of the sleep session and/or any additional portions. The one or more parameters of the respiratory therapy system may include motor speed, pressure, or both.

In some implementations, at step 1954, an alarm, a notification, or an instruction is generated based at least in part on the body position of the user identified at step 1940, and may be conveyed to the user via, for example, external device 170. Additionally or alternatively, in some implementations, at step 1956, the user of the respiratory therapy system is communicated with a recommended body position in another portion (e.g., a subsequent portion or a future portion) of the sleep session or another sleep session (e.g., a subsequent sleep session or a future sleep session) to aid in (i) reducing AHI, (ii) improving therapy, (iii) improving sleep quality, or (iv) any combination thereof.

In some implementations, at step 1958, the user of the respiratory therapy system is caused to change body position based at least in part on the body position of the user identified at step 1940 and/or the detection of respiratory events, e.g. apneas or hypopneas. In some examples, a smart pillow may be adjusted such that the smart pillow urges the user to change position of the user's head. In some examples, a smart bed or a smart mattress may be adjusted such that the smart bed or the smart mattress urges the user to change position of the user's body.

Additionally or alternatively, in some implementations, the action includes generating a report that correlates the identified body position of the user with (i) a sleep quality of the user, (ii) a therapy efficacy of the user, (iii) or both (i) and (ii). The body position of the user may be correlated with sleep quality and/or therapy efficacy for the portion(s) of the sleep session (and/or therapy session) that the user is in that body position, for example. Sleep quality may be expressed as a sleep score, and therapy efficacy as a therapy score, as described herein and may be correlated with the user position(s) during a sleep and/or therapy session or portion thereof.

Generally, the method 1900 can be implemented using a system having a control system with one or more processors, and a memory storing machine readable instructions. The controls system can be coupled to the memory; the method 1900 can be implemented when the machine readable instructions are executed by at least one of the processors of the control system. The method 1900 can also be implemented using a computer program product (such as a non-transitory computer readable medium) comprising instructions that when executed by a computer, cause the computer to carry out the steps of the method 1900.

While the system 100 and the method 1900 have been described herein with reference to a single user, more generally, the system 100 and the method 1900 can be used with a plurality of users simultaneously (e.g., two users, five users, 10 users, 20 users, etc.). For example, the system 100 and the method 1900 can be used in a cloud monitoring setting.

Additionally, or alternatively, in some implementations, the system 100 and/or the method 1900 can be used to monitor one or more patients while using one or more respiratory therapy systems (e.g., a respiratory therapy system described herein). For example, in some such implementations, a notification associated with movement event(s) and/or body position(s) associated with the one or more patients can be sent to a monitoring device or personnel. The movement event(s) and/or body position(s) can be determined using one or more steps of the method 1900. Additionally, or alternatively, in some implementations, the movement event(s) and/or body position(s) are simply being recorded.

One or more elements or aspects or steps, or any portion(s) thereof, from one or more of any of claims 1-153 below can be combined with one or more elements or aspects or steps, or any portion(s) thereof, from one or more of any of the other claims 1-153 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A system for identifying a body position of a user of a respiratory therapy system, the system comprising:
a sensor configured to generate airflow data associated with the user;
a memory storing machine-readable instructions; and
a control system including one or more processors configured to execute the machine-readable instructions to:
receive the airflow data associated with the user during a sleep session;
determine one or more features associated with the airflow data;
based at least in part on the determined one or more features, identify the body position of the user during a first portion of the sleep session; and
based at least in part on the identified body position of the user, cause an action to be performed.

2. (canceled)

3. The system of claim 1, wherein the airflow data includes flow rate data, pressure data, or both.

4. (canceled)

5. (canceled)

6. The system of claim 1, wherein the determined one or more features include one or more of a respiratory rate, an amplitude of flow rate signal, a change in amplitude of flow rate signal a number of apnea, a number of hypopnea events, a duration of an apnea event, a duration of a hypopnea event, a respiration rate, a change in respiration rate, an amplitude of flow rate signal, a change in amplitude of flow rate signal, a change in impulsiveness of flow rate signal, a spectral content of the flow rate signal, a randomness of the flow rate signal, a frequency content of flow rate signal a kurtosis flow rate signal, a spectral entropy of the flow rate signal, a variance of the flow rate signal, a variance of envelope of the flow rate signal, a root mean square of the flow rate signal, a median respiratory rate for the first portion of the sleep session, a standard deviation in respiratory rate for the first portion of the sleep session, a median amplitude of flow rate signal for the first portion of the sleep session, and a standard deviation in amplitude of the flow rate signal for the first portion of the sleep session.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The system of claim 1, wherein the determined one or more features include an amplitude of flow rate signal, wherein the control system is further configured to compare the amplitude of the flow rate signal in a range of fundamental frequency of human respiration with the amplitude of the flow rate signal at frequency ranges higher than the fundamental frequency of human respiration, and wherein the body position of the user is identified based at least in part on the comparison.

23. The system of claim 1, wherein the determined one or more features include a kurtosis of flow rate signal, a spectral entropy of the flow rate signal, a variance of the flow rate signal, a variance of envelope of the flow rate signal, a root mean square of the flow rate signal, a variance of the root mean square of the flow rate signal, or any combination thereof.

24. (canceled)

25. (canceled)

26. (canceled)

27. (canceled)

28. (canceled)

29. (canceled)

30. The system of claim 1, wherein the received airflow data is processed for the first portion of the sleep session to determine the one or more features associated with the user of the respiratory therapy system for the first portion of the sleep session.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. The system of claim 1, wherein the identifying the body position of the user of the respiratory therapy system includes comparing the determined one or more features to a plurality of historical features associated with a plurality of corresponding historical body positions.

45. The system of claim 44, wherein the plurality of historical features and the plurality of corresponding historical body positions are obtained from stored data associated with (i) the user of the respiratory therapy system, (ii) one or more other users of one or more other respiratory therapy systems, or (iii) both (i) and (ii).

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. A system for identifying a movement event of a user of a respiratory therapy system, the system comprising:
a sensor configured to generate airflow data associated with the user;
a memory storing machine-readable instructions; and
a control system including one or more processors configured to execute the machine-readable instructions to:
receive the airflow data associated with the user during a sleep session;
determine one or more features associated with the airflow data;
based at least in part on the determined one or more features, identify the movement event of the user during a first portion of the sleep session; and
based at least in part on the identified movement event of the user, cause an action to be performed.

76. The system of claim 75, wherein the control system is further configured to determine a body position of the user based at least in part on (i) the determined one or more features, (ii) the identified movement event of the user, or (iii) both (i) and (ii).

77. The system of claim 75, wherein the determined one or more features include a change in impulsiveness of flow rate signal, a spectral content of the flow rate signal, a randomness of the flow rate signal, or any combination thereof.

78. The system of claim 75, wherein the determined one or more features include a frequency content of flow rate signal, and wherein (i) a relative reduction in frequency content of the flow rate signal at or around a fundamental frequency of respiratory rate, (ii) a relative increase in the frequent content of the flow rate signal at higher frequencies than the fundamental frequency of respiratory rate, or (iii) both (i) and (ii) are indicative of a movement event and/or a change in body position.

79. (canceled)

80. (canceled)

81. (canceled)

82. The system of claim 75, wherein an increase in (i) an amplitude of flow rate signal, (ii) an impulsiveness of the flow rate signal, or (iii) both (i) and (ii) are indicative of a movement event and/or a change in body position.

83. The system of claim 75, wherein the determined one or more features include an amplitude of flow rate signal, wherein the control system is further configured to compare the amplitude of the flow rate signal in a range of fundamental frequency of human respiration with the amplitude of the flow rate signal at frequency ranges higher than the fundamental frequency of human respiration, and wherein the movement event of the user is identified based at least in part on the comparison.

84. (canceled)

85. (canceled)

86. (canceled)

87. (canceled)

88. (canceled)

89. (canceled)

90. The system of claim 75, wherein the determined one or more features include a kurtosis of flow rate signal, a spectral entropy of the flow rate signal, a variance of the flow rate signal, a variance of envelope of the flow rate signal, a root mean square of the flow rate signal, and a variance of the root mean square of the flow rate signal.

91. A method for identifying a body position of a user of a respiratory therapy system during a sleep session, the method comprising:
receiving airflow data associated with the user of the respiratory therapy system during the sleep session;
determining one or more features associated with the airflow data;
based at least in part on the determined one or more features, identifying the body position of the user during a first portion of the sleep session; and
based at least in part on the identified body position of the user, causing an action to be performed.

92. The method of claim 91, wherein the airflow data includes flow rate data, pressure data, or both.

93. The method of claim 91, wherein the determined one or more features include a respiratory rate, a change in respiratory rate, an amplitude of flow rate signal, a change in amplitude of the flow rate signal, a number of apnea events, a number of hypopnea events, a duration of apnea events, a duration of hypopnea events, or any combination thereof.

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. The method of claim 91, wherein the received airflow data is processed for the first portion of the sleep session to determine the one or more features associated with the user of the respiratory therapy system for the first portion of the sleep session.

106. (canceled)

107. The method of claim 91, wherein the first portion of the sleep session corresponds to a number of breaths, and wherein the number of breaths is one breath, two breaths, three breaths, four breaths, or five breaths.

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. The method of claim 91, wherein the identifying the body position of the user of the respiratory therapy system includes comparing the determined one or more features to a plurality of historical features associated with a plurality of corresponding historical body positions.

120. The method of claim 119, wherein the plurality of historical features and the plurality of corresponding historical body positions are obtained from stored data associated with (i) the user of the respiratory therapy system, (ii) one or more other users of one or more other respiratory therapy systems, or (iii) both (i) and (ii).

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)
